(19) United States
(12) Patent Application Publication
(10) Pub. No.: US 2007/0031835 A1
(43) Pub. Date: Feb. 8, 2007

(54) Diagnosis of advanced cancer

(76) Inventors: Gail Petuna Risbridger, Victoria (AU); Donna M Peehl, Mountain View, CA (US); Sally Louise Mellor, Victoria (AU); Emma Margaret Anne Ball, Victoria (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/551,352

(22) PCT Filed: Mar. 29, 2004
(86) PCT No.: PCT/AU04/00389

§ 371 Date: Oct. 4, 2006

Related U.S. Application Data
(60) Provisional application No. 60/457,939, filed on Mar. 28, 2003.

(51) Int. Cl. C12Q 1/68; G01N 33/574

(52) U.S. Cl. 435/6; 435/7.23

(57) ABSTRACT

The present invention relates generally to a method of diagnosing, predicting or monitoring the development or progress of advanced cancer and, in particular, to a method of diagnosing, predicting or monitoring the development of or progress of advanced prostate cancer in a mammal. The present invention contemplates a method for detecting advanced cancer, or predisposition to thereof, by screening for an increase in the level of inhibin expression in a mammal. The present invention further provides a method for diagnosing or monitoring conditions associated with or characterized by the onset of advanced cancer. Also provided are diagnostic agents useful for detecting inhibin or subunit thereof.

### FIELD OF THE INVENTION

The present invention relates generally to a method of diagnosing, predicting or monitoring the development or progress of advanced cancer and, in particular, to a method of diagnosing, predicting or monitoring the development of or progress of advanced prostate cancer in a mammal. The present invention contemplates a method for detecting advanced cancer, or predisposition to thereof, by screening for an increase in the level of inhibin expression in a mammal. The present invention further provides a method for diagnosing or monitoring conditions associated with or characterized by the onset of advanced cancer. The present invention further provides methods for the therapeutic or prophylactic treatment of conditions characterised by and advanced cancer.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge.

Prostate cancer is a disease that occurs in men mostly over the age of 50. It can occur in younger men but this is rare. Figures suggest that approximately one in four males above the age of 55 will suffer from a prostate disease of some form. The incidence in Australia of prostatic cancer is high and similarly prevalent rates occur in most countries. Globally, prostate cancer now represents the third highest incidence of cancer after lung cancer (due largely to smoking) and stomach cancer. This represents a significant cost to health care systems and decreases the quality of life of men suffering from this disorder.

Further, the incidence of prostate cancer appears to be increasing. This may be partly due to a ‘real’ increased risk, but is certainly related to the increased likelihood of detection, via PSA tests, and the increased number of TURPs operations. A TURP is done when prostate tissue is removed to improve symptoms of a benign prostate condition. However, in doing so, subsequent pathology sometimes indicates the existence of cancer.

Whether there is a real increase in risk or not, the numbers of cases of prostate cancer will rise due to the population at risk—older men—growing with the lengthening of life expectancy.

Although the causes of prostate cancer are not fully understood, men with a family history of prostate cancer in a first degree relative have two to three times the risk of developing the disease, indicating a role for genetic predisposition. However, the majority of prostate cancers are sporadic and unrelated to family history.

Caught early on, prostate cancer is usually a treatable disease. However, about half the men who are diagnosed with prostate cancer are unfortunately diagnosed at a late stage when the disease is less treatable. In this regard, early stage prostate cancer is generally localised to the prostate. Advanced prostate cancer, although having originated in the prostate, has generally spread beyond the prostate to other parts of the body signals a significantly less hopeful prognosis.

Prostate cancer pathologies are graded with a Gleason grading from 1 to 5 in order of increasing malignancy. Cribiform pathological patterns are sometimes observed at the stage of Gleason Grade 3 and 4. Cribiform pathologies are associated with prostate cancer progression and poor patient outcome.

Accordingly, although tests for diagnosing prostate cancer, per se, are currently available, a significant need exists to develop means of early detection of the transition to development of advanced prostate cancer, thereby providing the patient with the possibility of better tailored treatment regimes and potentially a significantly better prognosis.

Activins, composed of two β-subunits, β_{A} and/or β_{B}, and their antagonists the inhibins (combinations of an α- and either of the two β-subunits) are members of the transforming growth factor (TGF)-β superfamily [Vale et al. (1990) In Peptide growth factors and their receptors: Handbook of Experimental Physiology, Vol. 95 (Eds, Sporn, M. and Roberts, A.) Springer-Verlag, Berlin, pp. 211-248]. Activins regulate cell growth or differentiation by binding activin receptors and initiating a signaling cascade [Pangas et al. (2000), Trends Endocrinol Metab, 11, 309-314]. Changes in expression of inhibin/activin subunits, activin receptors, or the activin-binding proteins follistatin (FS315 and FS288) have been shown to influence growth of a variety of types of cells. Tumor suppressor activity of inhibin a in the gonads and adrenals has been recorded in transgenic mice bearing a targeted deletion of the inhibin a subunit [Matzuk et al. (1992), Nature, 360, 313-9; Matzuk et al. (1994), Semin Cancer Biol, 5, 37-45; Matzuk et al. (1994), Proc Natl Acad Sci USA, 91, 8817-21; Matzuk et al. (1996), Recent Prog Horm Res, 51, 123-54, 1996; Cipriano et al. (2000), Endocrinology, 141, 2319-27; Lopez et al. (1999), Oncogene, 18, 7303-9]. As a tumor suppressor, it was predicted that decreased expression of inhibin α may confer increased malignant potential. Specifically, it was demonstrated that methylation of the α-inhibin gene was observed more frequently in malignant tissues compared to normal tissues [Schmitt et al. (2002), Mol Endocrinol, 16, 213-20; Balanathan et al., Journal of Molecular Endocrinology, 32, 55-67, 2004].

Inhibin α and activin β subunits, and follistatins, are synthesized in the human prostate. Studies on transrectal ultrasound needle biopsies from 15 individuals with benign prostatic hyperplasia (13PH) and 12 patients with cancer, have shown a loss of inhibin α subunit mRNA by in situ hybridization and loss of protein expression based on immunolocalisation [Mellor et al. (1998), J Clin Endocrinol Metab, 83, 969-975.]. Using an immunopurified sheep polyclonal antibody αC41 against recombinant bovine inhibin a fusion protein and a polyclonal antibody αN320 against a fusion protein consisting of amino acids 1-26 of the α_{N} region of bovine inhibin α, staining in the non-malignant epithelium with both of these antibodies has been reported, but no staining in any of the 12 cancers with Gleason score 7-10. In subsequent studies, these observations have been confirmed and loss of inhibin a subunit expression has been determined to be due to LOH and/or promoter hypermethylation [Schmitt et al. (2002), Mol Endocrinol, 16, 213-20; Balanathan et al., 2004 supra].

Activin β_{A}- and β_{B}-subunits are also localized to the epithelium of benign tissues and poorly differentiated adenocarcinomas of the prostate [Thomas et al. (1997), J Clin Endocrinol Metab, 82, 3851-9; Thomas et al. (1998), Prostate, 34, 34-43]. Follistatin expression is noted in both benign epithelium and poorly differentiated cancer. Antibodies, raised to different isoforms, show distinct labelling patterns in malignant versus benign epithelia suggesting differential production by these prostatic compartments [Thomas 1997 TGFb, supra].

In work leading up to the present invention, it has been extremely surprisingly and unexpectedly found that advanced cancers, in general, are in fact, characterised by a significant increase in the levels of inhibin expression, in particular α-inhibin expression. These findings are extremely surprising in light of the extensive body of teaching that the inhibin gene acts as a tumour suppressor gene and the correlation, therefore, with previous diagnostic findings that cancer patients exhibited down-regulated levels of inhibin expression. These findings have now facilitated the development of a means for the highly selective detection of the onset of advanced cancer, in particular advanced prostate cancer. Also provided are means of monitoring the progress of advanced cancer and means of detecting the existence of a predisposition to developing advanced cancer. In the context of prostate cancer, the latter is particularly important to enable the identification and design of treatment regimes.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention is directed to a method of detecting the onset of an advanced neoplasm or a predisposition to developing an advanced neoplasm in a mammal said method comprising screening for the level of inhibin protein and/or gene expression in said mammal wherein an increase in the level of inhibin protein and/or gene expression is indicative of the onset of an advanced neoplasm or a predisposition to developing an advanced neoplasm.

In another embodiment, the present invention provides a method of detecting the onset of an advanced malignant neoplasm of the breast, thyroid, testis or adrenal gland or a predisposition to developing an advanced malignant neoplasm of the breast, thyroid, testis or adrenal gland in a mammal said method comprising screening for the level of inhibin, protein and/or gene expression in said mammal wherein an increase in the level of inhibin protein and/or gene expression is indicative of the onset of said advanced malignant neoplasm or a predisposition to developing said advanced malignant neoplasm.

In still another embodiment, the present invention provides a method of detecting the onset of an advanced malignant neoplasm of the oesophagus, stomach, colon, rectum, kidney, bladder, small intestine, large intestine, larynx, nasal cavity, throat,neural tissue or endometrium or a predisposition to developing an advanced malignant neoplasm of the oesophagus, stomach, colon, rectum, kidney, bladder, small intestine, large intestine, larynx, nasal cavity, throat,neural tissue or endometrium in a mammal said method comprising screening for the level of inhibin, protein and/or gene expression in said mammal wherein an increase in the level of inhibin protein and/or gene expression is indicative of the onset of said advanced malignant neoplasm or a predisposition to developing said advanced malignant neoplasm.

In yet still another preferred embodiment, the present invention provides a method of detecting the onset of an advanced malignant neoplasm of the cervix, brain, skin, lymph node, lung, salivary gland, liver, gallbladder or pancreas or a predisposition to developing an advanced malignant neoplasm of the cervix, brain, skin, lymph node, lung, salivary gland, liver, gallbladder or pancreas in a mammal said method comprising screening for the level of inhibin, protein and/or gene expression in said mammal wherein an increase in the level of inhibin protein and/or gene expression is indicative of the onset of said advanced malignant neoplasm or a predisposition to developing said advanced malignant neoplasm.

Another aspect of the present invention provides a method of detecting the onset of an advanced prostate malignant neoplasm or a predisposition to developing an advanced prostate malignant neoplasm in a mammal said method comprising screening for the level of inhibin, protein and/or gene expression in said mammal wherein an increase in the level of inhibin protein and/or gene expression is indicative of the onset of an advanced prostate malignant neoplasm or a predisposition to developing an advanced prostate malignant neoplasm.

Accordingly, the present invention provides a method of detecting the onset of advanced cancer of the breast, thyroid, testis or adrenal gland or a predisposition to developing advanced cancer of the breast, thyroid, testis or adrenal gland in a mammal said method comprising screening for the level of inhibin protein and/or gene expression in said mammal wherein an increase in the level of inhibin protein and/or gene expression is indicative of the onset of said advanced cancer or a predisposition to developing said advanced cancer.

Most preferably, said advanced cancer is metastatic cancer.

In another embodiment, the present invention provides a method of detecting the onset of advanced cancer of the oesophagus, stomach, colon, rectum, kidney, bladder, small intestine, large intestine, larynx, nasal cavity, throat,neural tissue or endometrium or a predisposition to developing advanced cancer of the oesophagus, stomach, colon, rectum, kidney, bladder, small intestine, large intestine, larynx, nasal cavity, throat,neural tissue or endometrium in a mammal said method comprising screening for the level of inhibin protein and/or gene expression in said mammal wherein an increase in the level of inhibin protein and/or gene expression is indicative of the onset of said advanced cancer or a predisposition to developing said advanced cancer.

Most preferably, said advanced cancer is metastatic cancer.

In yet another preferred embodiment, the present invention provides a method of detecting the onset of advanced cancer of the cervix, brain, skin, lymph node, lung, salivary gland, liver, gallbladder or pancreas or a predisposition to developing advanced cancer of cervix, brain, skin, lymph node, lung, salivary gland, liver, gallbladder or pancreas in a mammal said method comprising screening for the level of inhibin protein and/or gene expression in said mammal wherein an increase in the level of inhibin protein and/or gene expression is indicative of the onset of said advanced cancer or a predisposition to developing said advanced cancer.

Most preferably, said advanced cancer is metastatic cancer.

In yet another aspect the present invention provides a method of detecting the onset of advanced prostate cancer or a predisposition to developing advanced prostate cancer in a mammal said method comprising screening for the level of inhibin protein and/or gene expression in said mammal wherein an increase in the level of inhibin protein and/or gene expression is indicative of the onset of advanced prostate cancer or a predisposition to developing advanced prostate cancer.

The present invention therefore preferably provides a method of detecting the onset of advanced cancer of the breast, thyroid, testis or adrenal gland or a predisposition to developing advanced cancer of the breast, thyroid, testis or adrenal gland in a mammal said method comprising screening for the level of α-inhibin protein and/or gene expression in said mammal wherein an increase in the level of α-inhibin protein and/or gene expression is indicative of the onset of said advanced cancer or a predisposition to developing said advanced cancer.

Most preferably, said advanced cancer is metastatic cancer.

In another embodiment, the present invention provides a method of detecting the onset of advanced cancer of the oesophagus, stomach, colon, rectum, kidney, bladder, small intestine, large intestine, larynx, nasal cavity, throat,neural tissue or endometrium or a predisposition to developing advanced cancer of the oesophagus, stomach, colon, rectum, kidney, bladder, small intestine, large intestine, larynx, nasal cavity, throat,neural tissue or endometrium in a mammal said method comprising screening for the level of α-inhibin protein and/or gene expression in said mammal wherein an increase in the level of α-inhibin protein and/or gene expression is indicative of the onset of said advanced cancer or a predisposition to developing said advanced cancer.

Most preferably, said advanced cancer is metastatic cancer.

In yet another preferred embodiment, the present invention provides a method of detecting the onset of advanced cancer of the cervix, brain, skin, lymph node, lung, salivary gland, liver, gallbladder or pancreas or a predisposition to developing advanced cancer of cervix, brain, skin, lymph node, lung, salivary gland, liver, gallbladder or pancreas in a mammal said method comprising screening for the level of α-inhibin protein and/or gene expression in said mammal wherein an increase in the level of α-inhibin protein and/or gene expression is indicative of the onset of said advanced cancer or a predisposition to developing said advanced cancer.

Most preferably, said advanced cancer is metastatic cancer.

In still another aspect the present invention provides a method of detecting the onset of metastatic prostate cancer or a predisposition to developing metastatic prostate cancer in a mammal said method comprising screening for the level of inhibin protein and/or gene expression in said mammal wherein an increase in the level of inhibin protein and/or gene expression is indicative of the onset of metastatic prostate cancer or a predisposition to developing metastatic prostate cancer.

In yet still another aspect the present invention is directed to a method of detecting the onset of advanced prostate cancer or a predisposition to developing advanced prostate cancer in a mammal said method comprising screening for the level of α-inhibin protein and/or gene expression in said mammal wherein an increase in the level of α-inhibin protein and/or gene expression is indicative of the onset of advanced prostate cancer or a predisposition to developing advanced prostate cancer.

In still yet another aspect the present invention provides a method of detecting the onset of advanced prostate cancer or a predisposition to developing advanced prostate cancer in a mammal said method comprising screening for the level of α-inhibin in said mammal, which α-inhibin is characterised by amino acids 73-96 of the α-C region, wherein an increase in the level of said α-inhibin is indicative of the onset of advanced prostate cancer or a predisposition to developing advanced prostate cancer.

A further aspect of the present invention is directed to a method of monitoring for the onset or progression of an advanced neoplasm in a mammal said method comprising screening for the modulation in the level of inhibin in said mammal wherein the level of said inhibin relative to the normal level of inhibin is indicative of the onset or progression of an advanced neoplasm.

Another aspect of the present invention provides a diagnostic kit for assaying biological samples comprising an agent for detecting α-inhibin protein or encoding nucleic acid molecule and reagents useful for facilitating the detection by the agent in the first compartment. Further means may also be included, for example, to receive a biological sample. The agent may be any suitable detecting molecule.

Still, another aspect of the present invention is directed to a method of modulating the invasiveness of a cell, said method comprising modulating the level of intracellular inhibin protein.

Yet still another aspect of the present invention is directed to a method of modulating the invasiveness of a cell, said method comprising modulating the level of intracellular inhibin protein wherein up-regulating inhibin levels to a functionally effective level induces said invasiveness and down-regulating inhibin levels to a functionally ineffective level inhibits said invasiveness.

Still yet another aspect of the present invention is directed to a method of modulating the invasiveness of a neoplastic cell, said method comprising modulating the level of intracellular α-inhibin protein wherein up-regulating α-inhibin levels to a functionally effective level induces said invasiveness and down-regulating α-inhibin levels to a functionally ineffective level inhibits said invasiveness.

Accordingly, another aspect of the prevent invention is directed to a method of modulating the invasiveness of a neoplastic cell of the breast, thyroid, testis or adrenal gland, said method comprising modulating the level of intracellular α-inhibin wherein up-regulating α-inhibin levels to a functionally effective level induces said invasiveness and down-regulating α-inhibin levels to a functionally ineffective level inhibits said invasiveness.

Still, another aspect of the prevent invention is directed to a method of modulating the invasiveness of a neoplastic cell of the oesophagus, stomach, colon, rectum, kidney, bladder, small intestine, large intestine, larynx, nasal cavity, throat,neural tissue or endometrium, said method comprising modulating the level of intracellular α-inhibin wherein up-regulating α-inhibin levels to a functionally effective level induces said invasiveness and down-regulating α-inhibin levels to a functionally ineffective level inhibits said invasiveness.

Yet, another aspect of the prevent invention is directed to a method of modulating the invasiveness of a neoplastic cell of the cervix, brain, skin, lymph node, lung, salivary gland, liver, gallbladder or pancreas, said method comprising modulating the level of intracellular α-inhibin wherein up-regulating α-inhibin levels to a functionally effective level induces said invasiveness and down-regulating α-inhibin levels to a functionally ineffective level inhibits said invasiveness.

A further aspect of the present invention provides a method of modulating the invasiveness of a neoplastic prostate cell, said method comprising modulating the level of intracellular α-inhibin protein wherein up-regulating α-inhibin levels to a functionally effective level induces said invasiveness and down-regulating α-inhibin levels to a functionally ineffective level inhibits said invasiveness.

Another further aspect of the prevent invention is directed to a method of modulating the invasiveness of a neoplastic prostate cell, said method comprising modulating the level of intracellular α-inhibin wherein up-regulating α-inhibin levels to a functionally effective level induces said invasiveness and down-regulating α-inhibin levels to a functionally ineffective level inhibits said invasiveness.

In still another further aspect there is provided a method of down-regulating the invasiveness of a neoplastic prostate cell comprising down-regulating the level of intracellular α-inhibin functionally ineffective level.

In yet another further aspect there is provided a method of down-regulating the invasiveness of a neoplastic cell in a mammal, said method comprising administering to said mammal an effective amount of an agent for a time and under conditions sufficient to induce a functionally ineffective level of α-inhibin.

Still yet another further aspect of the present invention contemplates a method for the treatment and/or prophylaxis of a condition characterised by an advanced neoplasm or a predisposition to the development of a condition characterised by an advanced neoplasm in a mammal, said method comprising modulating the level of intracellular inhibin wherein down-regulating said inhibin levels to a functionally ineffective level inhibits invasiveness.

Yet still another further aspect of the present invention contemplates a method for the treatment and/or prophylaxis of a condition characterised by an advanced prostate neoplasm or a predisposition to the development of a condition characterised by an advanced prostate neoplasm in a mammal, said method comprising modulating the level of intracellular α-inhibin wherein down-regulating said α-inhibin levels to a functionally ineffective level inhibits prostate cell invasiveness.

Another aspect of the present invention contemplates a method for the treatment and/or prophylaxis of a condition characterised by an advanced prostate neoplasm or a predisposition to the development of a condition characterised by an advanced prostate neoplasm in a mammal, said method comprising administering the said mammal an effective amount of an agent for a time and under conditions sufficient to induce a functionally ineffective level of α-inhibin.

Still another aspect of the present invention relates to the use of an agent capable of modulating the functionally effective level of α-inhibin in the manufacture of a medicament for the treatment of an advanced neoplasm or a predisposition to developing an advanced neoplasm wherein down-regulating α-inhibin levels to a functionally ineffective level inhibits invasiveness.

In another aspect the present invention relates to the use of an aspect as hereinbefore described in the manufacture of a medicament for the regulation of the invasiveness of a neoplastic cell wherein down-regulating α-inhibin to a functionally ineffective level inhibits invasiveness.

In yet another further aspect, the present invention contemplates a pharmaceutical composition comprising the modulatory agent as hereinbefore defined and one or more pharmaceutically acceptable carriers and/or diluents. Said agents are referred to as the active ingredients

### BRIEF DESCRIPTION OF THE DRAWINGS

FIG. 1 is a graphical representation of the intensity scores of the inhibin a-12 staining in BPH and Pca.

FIG. 2 is a graphical representation of the effect on cellular growth rate when inhibin α is overexpressed in LNCaP cells. Growth curves for parental LNCaP, LNCaP clones transfected with inhibin α cDNA and LNCaP clones transfected with expression vector with no insert were established. Cells were seeded in equal amounts at day 0 and counted every day for consecutive 5 days. Closed grey symbols, parental LNCaP; open grey symbols, pcDNA3.1/GS transfected clones #16, #17 and #18; closed black symbols, inhibin α cDNA transfected clones #3 and #8. Overall, LNCaP cells overexpressing inhibin α (black) demonstrated slower rates of growth compared to LNCaP cells not expressing inhibin α (grey).

FIG. 3 is a graphical representation of the effect on cellular growth rate when inhibin a is overexpressed in PC3 cells. Growth curves for parental PC3, PC3 clones transfected with inhibin α cDNA and PC3 cells transfected with expression vector with no insert were established. Cells were seeded in equal amounts at day 0 and counted every day for consecutive 5 days. Closed grey symbols, parental PC3; open grey symbols, pcDNA3.1/GS transfected clones #128, #129 and #130; closed black symbols, inhibin a cDNA transfected clones #20, #103 and #104. Overall, PC3 cells overexpressing inhibin a (black) demonstrated increased rates of growth compared to PC3 cells not expressing inhibin α (grey).

FIG. 4 is a graphical representation of the effect on DNA synthesis when inhibin α is overexpressed in LNCaP cells. ³[M{ thymidine incorporation assays were established for parental LNCaP, LNCaP clones transfected with inhibin α cDNA and LNCaP clones transfected with expression vector only. The cells were seeded in equal amounts. After 2 days in culture, the cells were treated with ³[H] thymidine for 24 hrs. Open bars, parental LNCaP and pcDNA3.1/GS (pZeo)—transfected cells; closed black symbols, inhibin α cDNA transfected clones #3 and #8. p<0.05; ns, not significant. Overall, LNCaP cells overexpressing inhibin α (black bars) demonstrated a reduction in DNA synthesis compared to LNCaP cells not expressing inhibin α (open bars).

FIG. 5 is a graphical representation of the effect on DNA synthesis when inhibin α is overexpressed in PC3 cells. ³[H] thymidine incorporation assays were established for parental PC3, PC3 clones transfected with inhibin α cDNA and PC3 clones transfected with expression vector only. The cells were seeded in equal amounts. After 2 days in culture, the cells were treated with ³[H] thymidine for 24 hrs. Open bars, parental PC3 and pcDNA3.1/GS (pZeo) transfected cells; closed black symbols, inhibin α cDNA transfected clones #20, #103 and #104. p<0.05; ns, not significant. Overall, PC3 cells overexpressing inhibin α (black bars) demonstrated an increase in DNA synthesis compared to PC3 cells not expressing inhibin α (open bars).

FIG. 6 is a graphical representation of the difference in inhibin α expression in grade 3 and grade 4 prostate cancers. The intensity of immunohistochemical staining for inhibin a in Gleason grade 3 was compared to Gleason grade 4 prostate cancers within the same tissue section. Intensity was scored as no staining=1, variable±staining=2, 1+staining=3, 2+staining=4, 3+staining=5. Inhibin α staining significantly intensified as Gleason grade increased from grade 3 to grade 4 (p<0.05).

FIG. 7 shows the difference in inhibin α expression in cribriform prostate cancers compared to benign epithelium. The intensity of immunohistochemical staining for inhibin α in prostate tumours with cribriform pathology was compared to adjacent benign secretory epithelium. Intensity was scored as no staining=1, variable±staining=2, 1+staining=3, 2+staining=4, 3+staining=5. Benign secretory epithelium expressed inhibin a. Staining was significantly increased in cribriform cancers compared to benign epithelium (p<0.0005).

FIG. 8 is an image depicting the immunolocalisation of the inhibin α subunit in normal human pancreas and pancreatic adenocarcinoma with the monoclonal antibody, R1. Magnification in panel a and c is ×4 and magnification in panels b and d is ×40. Normal pancreas (a, b) was negative for inhibin α. In contrast, inhibin α was immunolocalised to the cytoplasm of tumour cells in the pancreatic adenocarcinoma tissue (c, d).

FIG. 9 is an image depicting the immunolocalisation of the inhibin α subunit in various normal human tissues and human cancers with the monoclonal antibody, PO12. Magnification in each panel is ×2 and is ×40 in the insets.

In normal skin (a, b) inhibin α immunolocalised to the cytoplasm of keratinocytes. In tissue from the patient with melanoma (c), the inhibin cc subunit strongly immunolocalised to the cytoplasm of tumour cells. In tissue from the patient with skin squamous cell carcinoma (d), the inhibin α subunit strongly immunolocalised to the cytoplasm of tumour cells.

Normal breast epithelium (e, f) strongly immunolocalised inhibin α subunit protein. Tissue from the patient with invasive papillary carcinoma of the breast displayed strong immunolocalisation of the inhibin α subunit in the cytoplasm of tumour tissue (g). Tissue from the patient with infiltrating breast carcinoma displayed weaker immunolocalisation of the inhibin α subunit in the cytoplasm of tumour tissue (h).

Inhibin α immunostaining was variable and weak in the normal lymph node tissue was negative for the inhibin α subunit protein (i, j). Normal lymph node tissue immunolocalised inhibin-α protein in some stromal tissue (j) surrounding the lymphocytes, however the lymphocytes themselves were negative for inhibin-α (i). Tissue from a patient with lymphoma displayed cytoplasmic staining for inhibin α in tumour cells (k).

In the normal lung, no staining for the inhibin α subunit protein was observed (l, m). Tissue from the patient with squamous cell carcinoma of the lung displayed immunolocalisation of the inhibin α subunit in the cytoplasm of tumour tissue (o). Tissue from the patient with lung adenocarcinoma displayed strong immunolocalisation of the inhibin α subunit in the cytoplasm of tumour tissue (p).

The normal salivary gland displayed immunostaining of the inhibin-α subunit protein in the cytoplasm of the ducts however no staining was observed in the serous or mucous cells (q). Tissue from the patient with pleomorphic adenoma of the parotid gland displayed strong immunostaining for inhibin α subunit in the cytoplasm of the tumour cells (r). Tissue from the patient with salivary duct carcinoma of the submandibular gland displayed strong immunostaining for inhibin α subunit in the cytoplasm of the tumour cells (s).

In the normal liver, hepatocytes strongly immunolocalised the inhibin α subunit protein (t, u). Tissue from the patient with hepatocellular carcinoma displayed strong immunolocalisation of inhibin α in the cytoplasm of tumour cells (v), although it was reduced compared to normal tissue.

In the normal gallbladder, basal and secretory cells localised the inhibin α subunit (x). Cytoplasmic staining was observed in the epithelial cell layer. Smooth muscle localisation was also observed. Tissue from a patient with adenocarcinoma of the gallbladder displayed strong cytoplasmic staining in the tumour cells (z). The inhibin α subunit immunolocalises to the cytoplasm of islet cells and the secretory granules of acinar cells in the normal pancreas (y). Tumour cells of pancreatic adenocarcinoma also display inhibin-α subunit staining (22).

FIG. 10 is an image depicting the immunolocalisation of the inhibin α subunit in various normal human tissues and human cancers with the monoclonal antibody, PO12. Magnification in each panel is ×2 and is ×40 in the insets.

In the normal esophagus (a, b) inhibin α demonstrated some nuclear staining in the epithelium. Tissue from a patient with squamous cell carcinoma of the esophagus strongly localised inhibin α subunit protein in the cytoplasm of the tumour cells (c).

Inhibin α subunit protein immunostaining was weak and variable in the body and antrum of the human stomach (d, e). In the body of the stomach some surface epithelial cells were positive and some nuclear staining was observed (d). In a patient with moderately differentiated stomach adenocarcinoma, a pattern of predominantly cytoplasmic staining was observed (f). A patient with poorly differentiated stomach adenocarcinoma displayed strong cytoplasmic staining (g).

The benign colon displayed moderately strong inhibin α subunit protein immunolocalisation in the surface epithelium (h). Tissue from a patient with adenocarcinoma of the colon displayed strong cytoplasmic staining in the tumour and moderate staining in the stroma (j). The normal rectum displayed variable staining of the surface epithelium and stroma (i). Rectal adenocarcinoma displayed moderately strong staining in tumour cells (k).

Normal kidney cortex displayed strong immunostaining for the inhibin-α subunit in the epithelium (l). Normal kidney medulla showed weak immunostaining in the epithelium (m). In contrast, both transitional cell carcinoma of the kidney (n) and renal cell carcinoma (o) showed strong immunostaining for inhibin α in tumour cells, although staining was stronger in the transitional cell carcinoma compared to renal cell carcinoma.

Normal urinary bladder stained moderately for inhibin α subunit in the epithelium and some stromal cells (p). Tissue from a patient with poorly differentiated carcinoma of the urinary bladder demonstrated strong immunostaining for inhibin α in the cytoplasm of tumour cells (q). Tissue from a patient with high grade transitional cell carcinoma of the urinary bladder demonstrated moderate staining for inhibin α in the cytoplasm of tumour cells (r).

Normal proliferative endometrium displayed some immunostaining for inhibin α in the stroma and moderately strong immunostaining in the epithelium (s). Normal human secretory endometrium displayed some immunostaining for inhibin α in the stroma and moderately strong immunostaining in the epithelium (t). Tissue from a patient with endometrial adenocarcinoma demonstrated strong staining for inhibin α in the cytoplasm of tumour cells (u).

FIG. 11 is an image depicting immunolocalisation of the inhibin α subunit in various normal human tissues and human cancers with the monoclonal antibody, PO12. Magnification in each panel is ×2 and is ×40 in the insets.

The uterine cervix displayed some cytoplasmic staining for inhibin α in the epithelium (a). Tissue from a patient with squamous cell carcinoma of the uterine cervix strongly immunolocalised the inhibin α subunit protein in the cytoplasm of tumour cells (b).

In the normal adrenal gland, strong inhibin α subunit protein was observed in the cytoplasm of some cells (c). Strong staining was also observed in tissues from patients with adrenal cortical carcinoma and adrenal pheochromocytoma (d and e, respectively).

In the normal thyroid, intermittent inhibin-α subunit staining was observed in the cytoplasm of the epithelial cells in the thyroid follicles (f). Inhibin alpha staining was positive in the nuclei of tumour cells in patients with thyroid papillary carcinoma (g) and displayed very strong cytoplasmic staining in minimally invasive follicular carcinoma of the thyroid (h).

Normal brain white matter was negative for inhibin α (i). In patients with meningioma, a primary tumour of the brain derived from meningiothelial cells, the inhibin-α subunit displayed strong staining in the cytoplasm of the meningioma cells (j).

The tubular cells of the human testis were strongly positive for inhibin α (k). Testis seminoma was also strongly positive for inhibin α (l).

### DETAILED DESCRIPTION OF THE INVENTION

The present invention is predicated, in part, on the surprising determination that advanced cancer is associated with an increase in the levels of α-inhibin expression. These findings have been made despite the prior art teachings that α-inhibin has a tumour suppressive role in cancer and has been found to be down-regulated, in terms of its level of expression. Accordingly, the correlation between increased α-inhibin levels and diagnosis of the onset or predisposition to the onset of advanced cancer, in particular advanced prostate cancer, has now facilitated the development of highly sensitive and informative means of diagnosing and/or predicting cancer progression.

Accordingly, one aspect of the present invention is directed to a method of detecting the onset of an advanced neoplasm or a predisposition to developing an advanced neoplasm in a mammal said method comprising screening for the level of inhibin protein and/or gene expression in said mammal wherein an increase in the level of inhibin protein and/or gene expression is indicative of the onset of an advanced neoplasm or a predisposition to developing an advanced neoplasm.

Reference to a “neoplasm” should be understood as a reference to an encapsulated or unencapsulated growth of neoplastic cells. Reference to a “neoplastic cell” should be understood as a reference to a cell exhibiting abnormal growth. The term “growth” should be understood in its broadest sense and includes reference to proliferation.

The phrase “abnormal growth” in this context is intended as a reference to cell growth which, relative to normal cell growth, exhibits one or more of an increase in the rate of cell division, an increase in the number of cell divisions, an increase in the length of the period of cell division, an increase in the frequency of periods of cell division or uncontrolled proliferation. Without limiting the present invention in any way, the common medical meaning of the term “neoplasia” refers to “new cell growth” that results as a loss of responsiveness to normal growth controls, eg. to neoplastic cell growth. Neoplasias include “tumours” which may be either benign, pre-malignant or malignant. The term “neoplasm” should be understood as a reference to a lesion, tumour or other encapsulated or unencapsulated mass or other form of growth which comprises neoplastic cells.

The term “neoplasm”, in the context of the present invention should be understood to include reference to all types of cancerous growths or oncogenic processes, metastatic tissues or malignantly transformed cells, tissues or organs irrespective of histopathologic type or state of invasiveness, where the tissue in issue expresses inhibin either constitutively or subsequently to an appropriate stimulus.

The neoplastic cells comprising the neoplasm may be any cell type, derived from any tissue, such as an epithelial or non-epithelial cell. Although the present invention is preferably directed to the diagnosis of advanced malignant neoplasms, the diagnosis and/or monitoring of non-malignant neoplasms is not excluded. In a preferred embodiment, the subject advanced neoplasm is an advanced neoplasm of the prostate, skin, breast, lymph node, lung, salivary gland, liver, gallbladder, pancreas, oesophagus, stomach, colon, rectum, kidney, bladder, endometrium, cervix, adrenal gland, thyroid, brain, small intestine, large intestine, larynx, nasal cavity, throat cancer, neural tumours or testis and even more preferably an advanced malignant neoplasm.

Accordingly, in one embodiment the present invention provides a method of detecting the onset of an advanced malignant neoplasm of the breast, thyroid, testis or adrenal gland or a predisposition to developing an advanced malignant neoplasm of the breast, thyroid, testis or adrenal gland in a mammal said method comprising screening for the level of inhibin, protein and/or gene expression in said mammal wherein an increase in the level of inhibin protein and/or gene expression is indicative of the onset of said advanced malignant neoplasm or a predisposition to developing said advanced malignant neoplasm.

Preferably, said neoplasm is a neoplasm of the breast.

In another embodiment, the present invention provides a method of detecting the onset of an advanced malignant neoplasm of the oesophagus, stomach, colon, rectum, kidney, bladder, small intestine, large intestine, larynx, nasal cavity, throat,neural tissue or endometrium or a predisposition to developing an advanced malignant neoplasm of the oesophagus, stomach, colon, rectum, kidney, bladder, small intestine, large intestine, larynx, nasal cavity, throat,neural tissue or endometrium in a mammal said method comprising screening for the level of inhibin, protein and/or gene expression in said mammal wherein an increase in the level of inhibin protein and/or gene expression is indicative of the onset of said advanced malignant neoplasm or a predisposition to developing said advanced malignant neoplasm.

In still another embodiment, the present invention provides a method of detecting the onset of an advanced malignant neoplasm of the cervix, brain, skin, lymph node, lung, salivary gland, liver, gallbladder or pancreas or a predisposition to developing an advanced malignant neoplasm of the cervix, brain, skin, lymph node, lung, salivary gland, liver, gallbladder or pancreas in a mammal said method comprising screening for the level of inhibin, protein and/or gene expression in said mammal wherein an increase in the level of inhibin protein and/or gene expression is indicative of the onset of said advanced malignant neoplasm or a predisposition to developing said advanced malignant neoplasm.

The present invention particularly provides a method of detecting the onset of an advanced prostate malignant neoplasm or a predisposition to developing an advanced prostate malignant neoplasm in a mammal said method comprising screening for the level of inhibin, protein and/or gene expression in said mammal wherein an increase in the level of inhibin protein and/or gene expression is indicative of the onset of an advanced prostate malignant neoplasm or a predisposition to developing an advanced prostate malignant neoplasm.

Reference to the subject neoplasm being “advanced” should be understood as a reference to the cancer metastasising or otherwise spreading beyond the organ or tissue in which is has originated. In the context of prostate cancer, this is beyond the capsule of the prostate organ. Without limiting the invention to any one theory or mode of action, metastases can generally form via distribution of the neoplastic cells through the bloodstream or the lymphatic channels or across body cavities such as the pleural or peritoneal spaces, thus setting up secondary tumours at sites distant from the original tumours. Any individual primary tumour will exhibit its own pattern of local behaviour and metastases. It should also be understood that reference to an “advanced” cancer encompasses any level or degree of spreading of the neoplastic cells beyond the organ or tissue in which it originated, whether that be relatively localised spreading in the immediate vicinity of the organ or tissue in issue or the more significant spreading of the neoplastic cells to other regions of the body, which accords with the more commonly understood notion of “metastatic” cancer. It also encompasses the form of cancer which is characterised by the development of metastases subsequently to removal of the organ or tissue in which the cancer originated. Accordingly, for example, “advanced” prostate cancer may develop, or a predisposition to development may be found to exist, subsequently to removal of the prostate. Reference to the terms “malignant neoplasm” and “cancer” herein should be understood to be interchangeable.

Accordingly, the present invention provides a method of detecting the onset of advanced cancer of the breast, thyroid, testis or adrenal gland or a predisposition to developing advanced cancer of the breast, thyroid, testis or adrenal gland in a mammal said method comprising screening for the level of inhibin protein and/or gene expression in said mammal wherein an increase in the level of inhibin protein and/or gene expression is indicative of the onset of said advanced cancer or a predisposition to developing said advanced cancer.

Preferably, said cancer is a neoplasm of the breast.

Most preferably, said advanced cancer is metastatic cancer.

In another embodiment, the present invention provides a method of detecting the onset of advanced cancer of the oesophagus, stomach, colon, rectum, kidney, bladder, small intestine, large intestine, larynx, nasal cavity, throat,neural tissue or endometrium or a predisposition to developing advanced cancer of oesophagus, stomach, colon, rectum, kidney, bladder, small intestine, large intestine, larynx, nasal cavity, throat,neural tissue or endometrium in a mammal said method comprising screening for the level of inhibin protein and/or gene expression in said mammal wherein an increase in the level of inhibin protein and/or gene expression is indicative of the onset of said advanced cancer or a predisposition to developing said advanced cancer.

Most preferably, said advanced cancer is metastatic cancer.

In yet another preferred embodiment, the present invention provides a method of detecting the onset of advanced cancer of the cervix, brain, skin, lymph note, lung, salivary gland, liver, gallbladder or pancreas or a predisposition to developing advanced cancer of cervix, brain, skin, lymph note, lung, salivary gland, liver, gallbladder or pancreas in a mammal said method comprising screening for the level of inhibin protein and/or gene expression in said mammal wherein an increase in the level of inhibin protein and/or gene expression is indicative of the onset of said advanced cancer or a predisposition to developing said advanced cancer.

Most preferably, said advanced cancer is- metastatic cancer.

Most preferably, the present invention provides a method of detecting the onset of advanced prostate cancer or a predisposition to developing advanced prostate cancer in a mammal said method comprising screening for the level of inhibin protein and/or gene expression in said mammal wherein an increase in the level of inhibin protein and/or gene expression is indicative of the onset of advanced prostate cancer or a predisposition to developing advanced prostate cancer.

Most preferably, said advance prostate cancer is metastatic prostate cancer.

According to this preferred embodiment, the present invention provides a method of detecting the onset of metastatic prostate cancer or a predisposition to developing metastatic prostate cancer in a mammal said method comprising screening for the level of inhibin protein and/or gene expression in said mammal wherein an increase in the level of inhibin protein and/or gene expression is indicative of the onset of metastatic prostate cancer or a predisposition to developing metastatic prostate cancer.

The present invention is predicated on the determination that increases in the level of expression of inhibin, in particular α-inhibin, are indicative of the development of advanced cancer, in particular advanced prostate cancer. In this regard, reference to “inhibin” should be read as including reference to all forms of inhibin and fragments, derivatives, mutants or variants thereof including all subunit polypeptides thereof including by way of example any protein encoded by the a subunit gene, the monomeric α-subunit polypeptide, the subunit precursor polypeptides pre, pro αN and αC, the dimeric αβ polypeptide (for example αβ_{A}, αβ_{B}, αβ_{C}, αβ_{D}, and αβ_{E}) and the dimeric precursor αC-β polypeptide.

Preferably, said inhibin is the α-subunit polypeptide (α-inhibin) or fragment, derivative, mutants or variants thereof. Reference to “α-inhibin” is not intended to be limiting and should be read as including reference to all forms of α-inhibin including any protein encoded by the α-subunit gene, all subunit polypeptides thereof including by way of example the monomeric subunit precursor polypeptides pre, pro αN and αC, and including, but not limited to, fragments, derivatives, mutants and variants thereof. Reference to “α-inhibin” also includes reference to any α-inhibin protein, whether existing as a monomer, multimer or fusion protein. It should also be understood to include reference to any isoforms which may arise from alternative splicing of α-inhibin mRNA or mutant or polymorphic variants of α-inhibin.

The present invention therefore preferably provides a method of detecting the onset of advanced cancer of the breast, thyroid, testis or adrenal gland or a predisposition to developing advanced cancer of the breast, thyroid, testis or adrenal gland in a mammal said method comprising screening for the level of α-inhibin protein and/or gene expression in said mammal wherein an increase in the level of α-inhibin protein and/or gene expression is indicative of the onset of said advanced cancer or a predisposition to developing said advanced cancer.

Preferably, said cancer is cancer of the breast.

Most preferably, said advanced cancer is metastatic cancer.

In another embodiment, the present invention provides a method of detecting the onset of advanced cancer of the oesophagus, stomach, colon, rectum, kidney, bladder, small intestine, large intestine, larynx, nasal cavity, throat,neural tissue or endometrium or a predisposition to developing advanced cancer of the oesophagus, stomach, colon, rectum, kidney, bladder, small intestine, large intestine, larynx, nasal cavity, throat,neural tissue or endometrium in a mammal said method comprising screening for the level of α-inhibin protein and/or gene expression in said mammal wherein an increase in the level of α-inhibin protein and/or gene expression is indicative of the onset of said advanced cancer or a predisposition to developing said advanced cancer.

Most preferably, said advanced cancer is metastatic cancer.

In yet another preferred embodiment, the present invention provides a method of detecting the onset of advanced cancer of the cervix, brain, skin, lymph node, lung, salivary gland, liver, gallbladder or pancreas or a predisposition to developing advanced cancer of cervix, brain, skin, lymph node, lung, salivary gland, liver, gallbladder or pancreas in a mammal said method comprising screening for the level of α-inhibin protein and/or gene expression in said mammal wherein an increase in the level of α-inhibin protein and/or gene expression is indicative of the onset of said advanced cancer or a predisposition to developing said advanced cancer.

Most preferably, said advanced cancer is metastatic cancer.

The present invention is most preferably directed to a method of detecting the onset of advanced prostate cancer or a predisposition to developing advanced prostate cancer in a mammal said method comprising screening for the level of α-inhibin protein and/or gene expression in said mammal wherein an increase in the level of α-inhibin protein and/or gene expression is indicative of the onset of advanced prostate cancer or a predisposition to developing advanced prostate cancer.

Preferably said advanced prostate cancer is metastatic prostate cancer.

The α-inhibin proteins which are detectable in the tissues from patients, in particular the prostate from patients diagnosed with benign prostate hyperplasia or in the non-malignant regions of prostate may comprise for example, are the αN and/or αC regions. The present invention is exemplified, but not limited in any way, by reference to detection of α-inhibin levels via the detection of the αC regions of the α-inhibin protein. α-inhibin proteins comprising αN and/or αC regions are also referred to as precursor α-subunit proteins. The αN and/or αC regions of precursor α-subunit proteins are found to exist either as part of an existing precursor α-subunit protein or in isolation, for example, following cleavage of said region from a precursor α-subunit protein. Precursor α-subunit proteins exist in many forms including, but not limited to, the forms pre- pro-αN -αC and pro-αC. According to this embodiment of the present invention, detection of α-inhibin proteins, including precursor α-subunit proteins, includes the detection of the αN and/or αC regions both in isolation, and as part of one or more of the various forms of precursor α-subunit protein.

Without limiting the present invention to any one theory or mode of action, it is thought that α-inhibin may undergo different forms of processing and/or cleavage at the α-C region of the α-inhibin subunit. This has been evidenced by the fact that the commonly used diagnostic antibody Groome R1 [Robertson et al., Mol Cell Endo. 180: 79-86], which is directed to the α-inhibin subunit amino acids 3-24 of the α-C region is unable to detect the presence of the form of α-inhibin which is increased in prostate biopsy samples exhibiting the onset of advanced cancer whereas the monoclonal antibody PO#12 [Robertson et al., Mol Cell Endo. 180: 79-86], directed to α-inhibin amino acids 73-96 of the α-C region, did detect these increased levels of α-inhibin.

Accordingly, in a preferred embodiment the form of α-inhibin which is detected is the form of α-inhibin which comprises amino acids 73-96 of the α-C region.

The present invention therefore preferably provides a method of detecting the onset of advanced cancer or a predisposition to developing advanced cancer in a mammal said method comprising screening for the level of α-inhibin in said mammal, which α-inhibin is characterised by amino acids 73-96 of the α-C region, wherein an increase in the level of said α-inhibin is indicative of the onset of advanced cancer or a predisposition to developing advanced cancer.

Most preferably, said α-inhibin protein is detected utilising the PO#12 monoclonal antibody and said advanced cancer is metastatic cancer.

The term “mammal” as used herein includes humans, primates, livestock animals (eg. horses, cattle, sheep, pigs, donkeys), laboratory test animals (eg. mice, rats, guinea pigs), companion animals (eg. dogs, cats) and captive wild animals (eg. kangaroos, deer, foxes). Preferably, the mammal is a human or a laboratory test animal. Even more preferably, the mammal is a human.

The present invention is predicated on the finding that levels of α-inhibin expression are modulated in malignant neoplastic tissue as compared to normal tissue or non-malignant neoplastic tissue. In this regard, the person of skill in the art will understand that one may screen for changes to α-inhibin levels at either the protein or the encoding nucleic acid molecule level. To the extent that it is not always specified, reference herein to screening for the level of “α-inhibin ” should be understood to include reference to screening for either the α-inhibin protein or its encoding primary RNA transcript or mRNA. Accordingly, it should be understood that the present invention is directed to the correlation of the level of α-inhibin relative to normal levels of this molecule. The “normal” level is the level of α-inhibin protein or encoding nucleic acid molecule in a biological sample corresponding to the sample being analysed of an individual who has not developed advanced cancer nor is predisposed to developing advanced cancer. The “normal” level also includes reference to the level of α-inhibin in non-malignant regions of the tissue which is the subject of analysis. This latter method of analysis is a relative form of analysis in terms of the normal and test levels being determined from non-neoplastic and test tissues, respectively, derived from a single individual. However, the method of the present invention should also be understood to encompass non-relative analyses means such as the analysis of test results relative to a standard result which reflects individual or collective results obtained from healthy individuals, other than the patient in issue. Said “normal level” may be a discrete level or a range of levels. Individuals exhibiting α-inhibin levels higher than the normal range are generally regarded as having undergone the onset of an advanced neoplasm or may be predisposed to the onset of an advanced neoplasm. In this regard, it should be understood that α-inhibin levels may be assessed or monitored by either quantitative or qualitative readouts. The reference level may also vary between individual forms (such as differently processed forms) of α-inhibin molecules.

Accordingly, the terms “increase” and “modulation” refer to increases and decreases in α-inhibin levels relative either to a normal reference level (or normal reference level range) or to an earlier result determined from the patient in issue, this latter reference point being particularly relevant in the context of the ongoing monitoring of a patient, as hereinafter described.

Without limiting the present invention to any one theory or mode of action, it is proposed that α-inhibin exhibits dual roles in carcinogenesis as both a tumour suppressor and prometastatic protein. The findings described herein support the concept of α-inhibin exhibiting a tumour suppressive role in cancer while also acting to promote metastatic disease in the context of cancer. It is thought that in the progression from confined prostate cancer to highly invasive tumour growth, the switch in α-inhibin action occurs. Reference to the “onset” of an advanced neoplasm, in particular advance prostate cancer, should be understood as a reference to one or more cells of that individual exhibiting abnormal growth characteristic. In this regard, the advanced neoplasm may be well developed in that a mass of proliferating cells has developed. Alternatively, the advanced neoplasm may be at a very early stage in that only relatively few divisions of the cells characterising the advanced neoplasm have occurred at the time of diagnosis. Nevertheless, the method of the present invention facilitates the identification of increased α-inhibin expression in these cells and, therefore, their detection. As detailed hereinbefore, it has also been determined that an increase in α-inhibin expression in neoplastic tissue correlates to the future development of an advanced neoplastic condition such as metastatic cancer, that is, before evidence of metastases formation occurs. Accordingly, the present invention also extends to the assessment of an individual's predisposition to the development of an advanced neoplasm, such as an advanced prostate cancer. Without limiting the present invention in any way, increased levels of α-inhibin in an individual who has not undergone the onset of advanced or high grade cancer development may be indicative of that individual's predisposition to developing an advanced neoplastic condition, such as the imminent development of an advanced cancer.

Although the preferred method is to detect an increase in α-inhibin levels in order to diagnose the onset of or predisposition to the onset of advanced cancer, the detection of decreases in α-inhibin levels may be desired under certain circumstances. For example, in the context of prostate cancer, where a radical prostatectomy is not performed, one may seek to monitor for changes in the disease state of the prostate, and its prognostic implications in relation to the development of advanced prostate cancers during the course of prophylactic or therapeutic treatment of the patients. Alternatively, patients presenting with symptoms of prostate disease or a genetic or environmental predisposition to the development of prostate disease may be monitored. In another example, to the extent that the prostate has been removed and analysis thereof has revealed a predisposition to the development of advanced prostate cancer (ie. the development of malignant prostate cell metastases), one may seek to monitor systemic or appropriately selected localised levels of α-inhibin as an indication of the development or regression of metastases. This aspect of the present invention therefore enables one to monitor the progression of an advanced cancer or predisposition thereto. It should be understood that in accordance with this aspect of the present invention, α-inhibin levels will likely be assessed relative to one or more previously obtained results, as hereinbefore described.

The method of the present invention is therefore useful as a one off test or as an on-going monitor of those individuals thought to be at risk of advanced neoplasm development or as a monitor of the effectiveness of therapeutic or prophylactic treatment regimes directed to inhibiting or otherwise slowing advanced neoplasm development. In these situations, mapping the modulation of α-inhibin in any one or more classes of biological samples is a valuable indicator of the status of an individual or the effectiveness of a therapeutic or prophylactic regime which is currently in use. Accordingly, the method of the present invention should be understood to extend to monitoring for increases or decreases in α-inhibin levels in an individual relative to their normal level (as hereinbefore defined) or relative to one or more earlier α-inhibin levels determined from said individual.

Accordingly, another aspect of the present invention is directed to a method of monitoring for the onset or progression of an advanced neoplasm in a mammal said method comprising screening for the modulation in the level of inhibin in said mammal wherein the level of said inhibin relative to the normal level of inhibin is indicative of the onset or progression of an advanced neoplasm.

Preferably, said advanced neoplasm is an advanced malignant prostate neoplasm.

In another preferred embodiment, said advanced neoplasm is an advanced malignant breast neoplasm.

In yet another preferred embodiment, said advanced neoplasm is an advanced neoplasm of the thyroid, testis or adrenal gland.

In still another preferred embodiment, said advanced neoplasm is an advanced malignant neoplasm or advanced cancer of the prostate, skin, breast, lymph node, lung, salivary gland, liver, gallbladder, pancreas, oesophagus, stomach, colon, rectum, kidney, bladder, small intestine, large intestine, larynx, nasal cavity, throat,neural tissue or endometrium or testis.

More preferably, said inhibin is α-inhibin and still more preferably the form of α-inhibin which comprises amino acids 73-96 of the αC region.

Still more preferably, said α-inhibin protein is detected utilising the PO#12 monoclonal antibody.

The method of the present invention has widespread applications including, but not limited to, the diagnostic or prognostic analysis of advanced cancer in particular advanced prostate cancer, or any condition characterised by the presence of advanced cancer, for example, the conditions associated with advanced prostate cancer such as urine retention, haematuria, urinary incontinence, kidney failure, bone pain, bone fragility, spinal cord damage, osteoarritis, lethargy, loss of appetite, nausea, diarrhea, constipation or cachexia.

Means of screening for changes in α-inhibin levels in an individual, or biological sample derived therefrom, can be achieved by any suitable method, which would be well known to the person of skill in the art, such as but not limited to:
(i) In vivo detection of α-inhibin. Molecular Imaging may be used following administration of imaging probes or reagents capable of disclosing altered expression levels of the α-inhibin mRNA or protein expression product in the prostate tissues.

Molecular imaging [Moore et al., BBA, 1402:239-249, 1988; Weissleder et al. Nature Medicine, 6:351-355, 2000] is the in vivo imaging of molecular expression that correlates with the macro-features currently visualized using “classical” diagnostic imaging techniques such as X-Ray, computed tomography (CT), MRI, Positron Emission Tomography (PET) or endoscopy. Historically, detection of malignant tumor cells in a background of normal or hyperplastic benign tissue is often based on differences in physical properties between tissues, which are frequently minimal, resulting in low contrast resolution. Application of expression profiling will define the differences in “molecular properties” between cancer and normal tissues that arise as a result of malignant transformation.
(ii) Detection of up-regulation of mRNA expression in the cells by Fluorescent In Situ Hybridization (FISH), or in extracts from the cells by technologies such as Quantitative Reverse Transcriptase Polymerase Chain Reaction (QRTPCR) or Flow cytometric qualification of competitive RT-PCR products [Wedemeyer et al., W. Clinical Chemistry 48:9 1398-1405, 2002] or array technologies.

For example, a labelled polynucleotide encoding α-inhibin may be utilized as a probe in a Northern blot of an RNA extract obtained from a tissue. Preferably, a nucleic acid extract from the animal is utilized in concert with oligonucleotide primers corresponding to sense and antisense sequences of a polynucleotide encoding α-inhibin, or flanking sequences thereof, in a nucleic acid amplification reaction such as RT PCR, real time PCR or SAGE. A variety of automated solid-phase detection techniques are also appropriate. For example, a very large scale immobilized primer arrays (VLSIPS™) are used for the detection of nucleic acids as, for example, described by Fodor et al., 1991 and Kazal et al., 1996. The above genetic techniques are well known to persons skilled in the art.

For example, to detect α-inhibin encoding RNA transcripts, RNA is isolated from a cellular sample suspected of containing α-inhibin RNA, e.g. total RNA isolated from human prostate cancer tissue. RNA can be isolated by methods known in the art, e.g. using TRIZOL™ reagent (GIBCO-BRL/Life Technologies, Gaithersburg, Md.). Oligo-dT, or random-sequence oligonucleotides, as well as sequence-specific oligonucleotides can be employed as a primer in a reverse transcriptase reaction to prepare first-strand cDNAs from the isolated RNA. Resultant first-strand cDNAs are then amplified with sequence-specific oligonucleotides in PCR reactions to yield an amplified product.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers. These primers will be identical or similar in sequence to opposite strands of the template to be amplified. PCR can be used to amplify specific RNA sequences and cDNA transcribed from total cellular RNA. See generally Mullis et al., 1987; Erlich, 1989. Thus, amplification of specific nucleic acid sequences by PCR relies upon oligonucleotides or “primers” having conserved nucleotide sequences wherein the conserved sequences are deduced from alignments of related gene or protein sequences, e.g. a sequence comparison of mammalian α-inhibin genes. For example, one primer is prepared which is predicted to anneal to the antisense strand and another primer prepared which is predicted to anneal to the sense strand of a cDNA molecule which encodes α-inhibin.

To detect the amplified product, the reaction mixture is typically subjected to agarose gel electrophoresis or other convenient separation technique and the relative presence of the α-inhibin specific amplified DNA detected. For example, α-inhibin amplified DNA may be detected using Southern hybridization with a specific oligonucleotide probe or comparing is electrophoretic mobility with DNA standards of known molecular weight. Isolation, purification and characterization of the amplified α-inhibin DNA may be accomplished by excising or eluting the fragment from the gel (for example, see references Lawn et al., 1981; Goeddel et al., 1980), cloning the amplified product into a cloning site of a suitable vector, such as the pCRII vector (Invitrogen), sequencing the cloned insert and comparing the DNA sequence to the known sequence of α-inhibin. The relative amounts of α-inhibin mRNA and cDNA can then be determined.
(iii) Measurement of altered α-inhibin protein levels in cell extracts or blood or other suitable biological sample, either qualitatively or quantitatively, for example by immunoassay, utilising immunointeractive molecules such as the PO#12 antibody, to detect αβ dimers, a monomeric subunit and/or αC or αN isoform of the a monomeric subunit.

In one example, one may seek to detect α-inhibin -immunointeractive molecule complex formation. For example, an antibody according to the invention, having a reporter molecule associated therewith, may be utilized in immunoassays. Such immunoassays include but are not limited to radioimmunoassays (RIAs), enzyme-linked immunosorbent assays (ELISAs) and immunochromatographic techniques (ICTs), Western blotting which are well known to those of skill in the art. For example, reference may be made to “Current Protocols in Immunology”, 1994 which discloses a variety of immunoassays which may be used in accordance with the present invention. Immunoassays may include competitive assays. It will be understood that the present invention encompasses qualitative and quantitative immunoassays.

Suitable immunoassay techniques are described, for example, in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen. The antigen in this case is α-inhibin or a fragment thereof.

Two-site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilized first antibody.

An alternative method involves immobilizing the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

From the foregoing, it will be appreciated that the reporter molecule associated with the antigen-binding molecule may include the following:
(a) direct attachment of the reporter molecule to the antibody;
(b) indirect attachment of the reporter molecule to the antibody; i.e., attachment of the reporter molecule to another assay reagent which subsequently binds to the antibody; and
(c) attachment to a subsequent reaction product of the antibody.

The reporter molecule may be selected from a group including a chromogen, a catalyst, an enzyme, a fluorochrome, a chemiluminescent molecule, a paramagnetic ion, a lanthanide ion such as Europium (Eu³⁴), a radioisotope including other nuclear tags and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in U.S. Pat. No. 4,366,241, U.S. Pat. No. 4,843,000, and U.S. Pat. No. 4,849,338. Suitable enzymes useful in the present invention include alkaline phosphatase, horseradish peroxidase, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al., International Publication No. WO 93/06121. Reference also may be made to the fluorochromes described in U.S. Pat. No. 5,573,909 [Singer et all, U.S. Pat. No. 5,326,692 [Brinkley et al]. Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognized, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art and are particularly useful for the present method. However, other reporter molecules, such as radioisotope, chemiluminescent or bioluminescent molecules may also be employed.
(iv) The use of aptamers in screening for nucleic acid molecules or expression products
(v) Determining altered protein expression based on any suitable functional test, enzymatic test or immunological test in addition to those detailed in point (iii) above.

As detailed above, any suitable technique may be utilised to detect α-inhibin or its encoding nucleic acid molecule. The nature of the technique which is selected for use will largely determine the type of biological sample which is required for analysis.

Reference to a “biological sample” should be understood as a reference to any sample of cells or tissue which is derived from an organism. The cells may be single cells, cultured cells or part of a tissue. In this regard, the biological sample may be derivable from any human or non-human mammal, as detailed above. It should be further understood that reference to “organism” includes reference to embryos and foetuses.

The biological sample may be any sample of material derived from the organism. This includes reference to both samples which are naturally present in the organism, such as tissue and body fluids in a mammal (for example biopsy specimens such as lymphoid specimens, resected tissue, tissue extracts, blood, lymph fluid, faeces, bronchial secretions or cell culture medium) and samples which are introduced into the body of the organism and subsequently removed, such as, for example, the saline solution extracted from the lung following a lung lavage or from the colon following an enema. It also includes reference to cells which originated from an organism but have been maintained in vitro, for example cell lines, or which have been manipulated or treated subsequently to removal from the organism, for example immortalised or genetically modified cells or tissues.

The biological sample which is tested according to the method of the present invention may be tested directly or may require some form of treatment prior to testing. For example, a biopsy sample may require homogenisation prior to testing. Where the sample comprises cellular material, it may be necessary to extract or otherwise expose the nucleic acid material present in the cellular material in order to facilitate analysis of the nucleic acid material in terms of its mRNA expression, for example. In yet another example, the sample may be partially purified or otherwise enriched prior to analysis. For example, to the extent that a biological sample comprises a very diverse cell population, it may be desirable to select out a sub-population of particular interest.

The choice of what type of sample is most suitable for testing in accordance with the method disclosed herein will be dependent on the nature of the condition which is being monitored. For example, if the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one organism.

Although the method of the present invention is most conveniently performed by analysis of an isolated biological sample, it should also be understood that reference to analysing a sample “derived from” a mammal includes reference to analysing the sample in vivo.

Another aspect of the present invention provides a diagnostic kit for assaying biological samples comprising an agent for detecting inhibin protein or encoding nucleic acid molecule and reagents useful for facilitating the detection by the agent in the first compartment. Further means may also be included, for example, to receive a biological sample. The agent may be any suitable detecting molecule.

Preferably, said inhibin is α-inhibin.

More preferably, said agent is the monoclonal antibody PO#12.

In a related aspect, the findings of the present invention now facilitate the development of methodology directed to the therapeutic and prophylactic treatment of conditions characterised by the development, and predisposition to the development of an advanced neoplasm, such as metastatic prostate cancer. These methods are based on the notion of down-regulating inhibin levels, in particular α-inhibin levels, thereby down-regulating the invasive potential of metastatic neoplastic cells.

Accordingly, another aspect of the present invention is directed to a method of modulating the invasiveness of a cell, said method comprising modulating the level of intracellular inhibin protein.

More particularly, the present invention is directed to a method of modulating the invasiveness of a cell, said method comprising modulating the level of intracellular inhibin protein wherein up-regulating inhibin levels to a functionally effective level induces said invasiveness and down-regulating inhibin levels to a functionally ineffective level inhibits said invasiveness.

Most particularly, said inhibin is α-inhibin.

Even more particularly, the present invention is directed to a method of modulating the invasiveness of a neoplastic cell, said method comprising modulating the level of intracellular α-inhibin protein wherein up-regulating α-inhibin levels to a functionally effective level induces said invasiveness and down-regulating α-inhibin levels to a functionally ineffective level inhibits said invasiveness.

Reference to “invasiveness” should be understood as a reference to the functional capacity of a neoplastic cell to metastasise or otherwise spread beyond the organ or tissue in which that cell originated, to other organs or tissues. In this regard, reference to “neoplastic cell” should be understood to have the same meaning as hereinbefore defined. Similarly, the issue of metastitisation, particularly in respect of prostate cancer, has been hereinbefore described.

Reference to “inhibin” and “α-inhibin ” should be understood as having the same meaning as hereinbefore defined but additionally includes reference to homologues thereof. Preferably, said α-inhibin is the α-inhibin subunit as hereinbefore defined.

Accordingly, another aspect of the prevent invention is directed to a method of modulating the invasiveness of a neoplastic cell of the breast, thyroid, testis or adrenal gland, said method comprising modulating the level of intracellular α-inhibin wherein up-regulating α-inhibin levels to a functionally effective level induces said invasiveness and down-regulating α-inhibin levels to a functionally ineffective level inhibits said invasiveness.

Still, another aspect of the prevent invention is directed to a method of modulating the invasiveness of a neoplastic cell of the oesophagus, stomach, colon, rectum, kidney, bladder, small intestine, large intestine, larynx, nasal cavity, throat,neural tissue or endometrium, said method comprising modulating the level of intracellular α-inhibin wherein up-regulating α-inhibin levels to a functionally effective level induces said invasiveness and down-regulating α-inhibin levels to a functionally ineffective level inhibits said invasiveness.

Yet another aspect of the prevent invention is directed to a method of modulating the invasiveness of a neoplastic cell of the cervix, brain, skin, lymph node, lung, salivary gland, liver, gallbladder or pancreas, said method comprising modulating the level of intracellular α-inhibin wherein up-regulating α-inhibin levels to a functionally effective level induces said invasiveness and down-regulating α-inhibin levels to a functionally ineffective level inhibits said invasiveness.

Most preferably, another aspect of the prevent invention is directed to a method of modulating the invasiveness of a neoplastic prostate cell, said method comprising modulating the level of intracellular α-inhibin wherein up-regulating α-inhibin levels to a functionally effective level induces said invasiveness and down-regulating α-inhibin levels to a functionally ineffective level inhibits said invasiveness.

Reference to “modulating” should be understood as a reference to up-regulating or down-regulating the subject invasiveness. Reference to “down-regulating” invasiveness should therefore be understood as a reference to preventing, reducing (eg. slowing) or otherwise inhibiting one or more aspects of invasiveness while reference to “up-regulating” should be understood to have the converse meaning. Although the preferred method is to down-regulate the invasiveness of a metastatic cell or to prevent the shift to a metastatic form of a cell predisposed in this regard, the present invention nonetheless extends to up-regulating the invasiveness, in particular the metastatic potential of a neoplastic cell, which may be desired in certain circumstances. For example, one may seek to utilise the method of the present invention to induce a metastatic cellular phenotype in vitro in order to enable one to screen for other potential therapeutic means.

The present invention therefore preferably provides a method of down-regulating the invasiveness of a neoplastic cell comprising down-regulating the level of intracellular α-inhibin functionally ineffective level.

Reference herein to attaining either a “functionally effective level” or “functionally ineffective level” of α-inhibin protein should be understood as a reference to attaining that level of α-inhibin at which modulation of neoplastic cell invasiveness can be achieved, whether that be up-regulation or down-regulation. In this regard, it is within the skill of the person of skill in the art to determine, utilising routine procedures, the threshold level of α-inhibin expression above which invasiveness is induced and below which invasiveness is prevented.

It should be understood that reference to an “effective level” means the level necessary to at least partly attain the desired response. The amount may vary depending on the health and physical condition of the cellular population and/or individual being treated, the taxonomic group of the cellular population and/or individual being treated, the degree of up or down-regulation which is desired, the formulation of the composition which is utilised, the assessment of the medical situation and other relevant factors. Accordingly, it is expected that this level may vary between individual situations, thereby falling in a broad range, which can be determined through routine trials.

Modulating α-inhibin levels may be achieved by any suitable means including, but not limited to:
(i) Modulating absolute levels of α-inhibin such that either more or less α-inhibin is present in the intracellular environment.
(ii) Agonising or antagonising α-inhibin protein functional activity such that the functional effectiveness of α-inhibin is either increased or decreased. For example, increasing the half life of α-inhibin may achieve an increase in the functionally effective level of α-inhibin without actually necessitating an increase in the absolute intracellular concentration of α-inhibin. Similarly, the partial antagonism of α-inhibin may act to reduce, although not necessarily eliminate, the functional effectiveness of said α-inhibin.

Accordingly, this may provide a means of down-regulating α-inhibin functioning without necessarily down-regulating absolute concentrations of α-inhibin.

In terms of achieving the up or down-regulation of α-inhibin, means for achieving this objective would be well known to the person of skill in the art and include, but are not limited to:
(i) Introducing into a cell a nucleic acid molecule encoding α-inhibin or in order to up-regulate the capacity of said cell to express α-inhibin.
(ii) Introducing into a cell a proteinaceous or non-proteinaceous molecule which modulates transcriptional and/or translational regulation of a gene, wherein this gene may be the α-inhibin gene or functional portion thereof or some other gene or gene region (eg. promoter region) which directly or indirectly modulates the expression of the α-inhibin gene.
(iii) Introducing into a cell the α-inhibin expression product.
(iv) Introducing a proteinaceous or non-proteinaceous molecule which functions as an antagonist to the α-inhibin expression product.
(v) Introducing a proteinaceous or non-proteinaceous molecule which functions as an agonist of the α-inhibin expression product.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the α-inhibin expression product or small molecules capable of acting as agonists or antagonists. Chemical agonists may not necessarily be derived from the α-inhibin expression product but may share certain conformational similarities. Alternatively, chemical agonists may be specifically designed to meet certain physiochemical properties. Antagonists may be any compound capable of blocking, inhibiting or otherwise preventing α-inhibin from carrying out its normal biological function. Antagonists include monoclonal antibodies and antisense nucleic acids which prevent transcription or translation of α-inhibin genes or mRNA in mammalian cells. Modulation of expression may also be achieved utilising antigens, RNA, ribosomes, DNAzymes, aptamers, antibodies (for example, the PO#12 monoclonal antibody) or molecules suitable for use in cosuppression. The proteinaceous and non-proteinaceous molecules referred to in points (i)-(v), above, are herein collectively referred to as “modulatory agents”.

Screening for the modulatory agents hereinbefore defined can be achieved by any one of several suitable methods including, but in no way limited to, contacting a cell comprising the α-inhibin gene or functional equivalent or derivative thereof with an agent and screening for the modulation of α-inhibin protein production or functional activity, modulation of the expression of a nucleic acid molecule encoding α-inhibin or modulation of the activity or expression of a downstream α-inhibin cellular target. Detecting such modulation can be achieved utilising techniques such as Western blotting, electrophoretic mobility shift assays and/or the readout of reporters of et-inhibin activity such as luciferases, CAT and the like.

It should be understood that the α-inhibin gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate α-inhibin activity, at either the nucleic acid or expression product levels, or the gene may require activation—thereby providing a model useful for, inter alia, screening for agents which up-regulate α-inhibin expression. Further, to the extent that an α-inhibin nucleic acid molecule is transfected into a cell, that molecule may comprise the entire α-inhibin gene or it may merely comprise a portion of the gene such as the portion which regulates expression of the α-inhibin product. For example, the α-inhibin promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively. In another example, the subject of detection could be a downstream α-inhibin regulatory target, rather than α-inhibin itself. Yet another example includes α-inhibin binding sites ligated to a minimal reporter. Modulation of α-inhibin activity can be detected by screening for the modulation of cellular invasiveness. This is an example of an indirect system where modulation of α-inhibin expression, per se, is not the subject of detection. Rather, modulation of the down-stream activity which α-inhibin regulates is monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the α-inhibin nucleic acid molecule or expression product itself or which modulate the expression of an upstream molecule, which upstream molecule subsequently modulates α-inhibin expression or expression product activity. Accordingly, these methods provide a mechanism of detecting agents which either directly or indirectly modulate α-inhibin expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, said agent is associated with a molecule which permits its targeting to a localised region.

The subject proteinaceous or non-proteinaceous molecule may act either directly or indirectly to modulate the expression of α-inhibin or the activity of the α-inhibin expression product. Said molecule acts directly if it associates with the α-inhibin nucleic acid molecule or expression product to modulate expression or activity, respectively. Said molecule acts indirectly if it associates with a molecule other than the α-inhibin nucleic acid molecule or expression product which other molecule either directly or indirectly modulates the expression or activity of the α-inhibin nucleic acid molecule or expression product, respectively. Accordingly, the method of the present invention encompasses the regulation of α-inhibin nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule. Reference to “modulation” should be understood as a reference to up-regulation or down-regulation.

“Derivatives” of the molecules herein described (for example α-inhibin or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, α-inhibin or derivative thereof may be fused to a molecule to facilitate its entry into a cell. Analogs of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogs.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” or “mutant” of α-inhibin should be understood to mean molecules which exhibit at least some of the functional activity of the form of α-inhibin of which it is a variant or mutant. A variation or mutation may take any form and may be naturally or non-naturally occurring.

A “homologue” is meant that the molecule is derived from a species other than that which is being treated in accordance with the method of the present invention. This may occur, for example, where it is determined that a species other than that which is being treated produces a form of α-inhibin which exhibits similar and suitable functional characteristics to that of the α-inhibin which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening. Antagonistic agents can also be screened for utilising such methods.

For example, libraries containing small organic molecules may be screened, wherein organic molecules having a large number of specific parent group substitutions are used. A general synthetic scheme may follow published methods [eg., Bunin B A, et al. (1994) Proc. Natl. Acad. Sci. USA, 91:4708-4712; DeWitt S H, et al. (1993) Proc. Natl. Acad. Sci. USA, 90:6909-6913]. Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing α-inhibin analogues which exhibit properties such as more potent pharmacological effects. α-inhibin or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of α-inhibin one may identify and utilise molecules which function agonistically or antagonistically to α-inhibin in order to up or down-regulate the functional activity of α-inhibin in relation to modulating cellular invasiveness. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of α-inhibin capable of acting as agonists or antagonists. Chemical agonists may not necessarily be derived from α-inhibin but may share certain conformational similarities. Alternatively, chemical agonists may be specifically designed to mimic certain physiochemical properties of α-inhibin. Antagonists may be any compound capable of blocking, inhibiting or otherwise preventing α-inhibin from carrying out its normal biological functions. Antagonists include monoclonal antibodies specific for α-inhibin or parts of α-inhibin.

Analogues of α-inhibin or of α-inhibin agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The method of the present invention contemplates the modulation of cellular invasiveness both in vitro and in vivo. Although the preferred method is to treat an individual in vivo it should nevertheless be understood that it may be desirable that the method of the invention may be applied in an in vitro environment, for example to facilitate the generation of metastatic cell lines. In another example, the application of the method of the present invention in an in vitro environment may extend to providing a read out mechanism for screening technologies such as those hereinbefore described. That is, molecules identified utilising these screening techniques can be assayed to observe the extent and/or nature of their functional effect on cellular invasiveness.

Modulation of said α-inhibin functional levels is achieved via the administration of α-inhibin a nucleic acid molecule encoding α-inhibin or an agent which effects modulation of α-inhibin activity or α-inhibin gene expression (herein collectively referred to as “modulatory agents”). Preferably, the subject method is utilised to down-regulate the cellular invasiveness of a neoplastic cell in a mammal.

Accordingly, in a preferred embodiment there is provided a method of down-regulating the invasiveness of a neoplastic cell in a mammal, said method comprising administering to said mammal an effective amount of an agent for a time and under conditions sufficient to induce a functionally ineffective level of α-inhibin.

Preferably, said neoplastic cell is a cell of the prostate, skin, breast, lymph node, lung, salivary gland, liver, gallbladder, pancreas, oesophagus, stomach, colon, rectum, kidney, bladder, endometrium, cervix, adrenal gland, thyroid, brain, small intestine, large intestine, larynx, nasal cavity, throat cancer or neural tumours or testis and even more preferably an advanced malignant neoplasm.

Reference to “induce” should be understood as a reference to achieving the desired α-inhibin level, whether that be a functionally effective level or a functionally ineffective level. Said induction is most likely to be achieved via the up-regulation or down-regulation of α-inhibin expression, as hereinbefore described, although any other suitable means of achieving induction are nevertheless herewith encompassed by the method of the present invention.

A further aspect of the present invention relates to the use of the invention in relation to the treatment and/or prophylaxis of disease conditions or other unwanted conditions. Without limiting the present invention to any one theory or mode of action, regulation of the invasiveness of a neoplastic cell, and in particular a neoplastic prostate cell provides a means of treating advanced forms of cancer by down-regulating the metastatic potential or the metastatic nature of the neoplastic cells which characterise the subject cancer. In particular, and without limiting the present invention to any one theory or mode of action, in addition to being an identifier of metastatic cells, the up-regulation of α-inhibin levels is also identifiable in cells which are predisposed to becoming invasive. Accordingly, “down-regulating” or “inhibiting” invasiveness includes reference to preventing the onset of invasiveness.

The present invention therefore contemplates a method for the treatment and/or prophylaxis of a condition characterised by an advanced neoplasm or a predisposition to the development of a condition characterised by an advanced neoplasm in a mammal, said method comprising modulating the level of intracellular inhibin wherein down-regulating said inhibin levels to a functionally ineffective level inhibits invasiveness.

Preferably, said inhibin is α-inhibin.

Preferably, said advanced neoplasm is an advanced neoplasm of the prostate, skin, breast, lymph node, lung, salivary gland, liver, gallbladder, pancreas, oesophagus, stomach, colon, rectum, kidney, bladder, endometrium, cervix, adrenal gland, thyroid, brain, small intestine, large intestine, larynx, nasal cavity, throat cancer or neural tumours or testis and even more preferably an advanced malignant neoplasm.

More particularly, the present invention contemplates a method for the treatment and/or prophylaxis of a condition characterised by an advanced prostate neoplasm or a predisposition to the development of a condition characterised by an advanced prostate neoplasm in a mammal, said method comprising modulating the level of intracellular α-inhibin wherein down-regulating said α-inhibin levels to a functionally ineffective level inhibits prostate cell invasiveness.

Still more preferably, the present invention contemplates a method for the treatment and/or prophylaxis of a condition characterised by an advanced prostate neoplasm or a predisposition to the development of a condition characterised by an advanced prostate neoplasm in a mammal, said method comprising administering the said mammal an effective amount of an agent for a time and under conditions sufficient to induce a functionally ineffective level of α-inhibin.

Preferably, said condition is advanced prostate cancer and, even more preferably, metastatic prostate cancer.

An “effective amount” means an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome. For example, one may combine the method of the present invention with radiotherapy directed to the primary tumour.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, said route of administration is oral.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention relates to the use of an agent capable of modulating the functionally effective level of α-inhibin in the manufacture of a medicament for the treatment of an advanced neoplasm or a predisposition to developing an advanced neoplasm wherein down-regulating α-inhibin levels to a functionally ineffective level inhibits invasiveness.

In another aspect the present invention relates to the use of an agent as hereinbefore described in the manufacture of a medicament for the regulation of the invasiveness of a neoplastic cell wherein down-regulating α-inhibin to a functionally ineffective level inhibits invasiveness.

According to these preferred embodiments, the subject neoplastic cells are preferably neoplastic cells of the prostate, skin, breast, lymph node, lung, salivary gland, liver, gallbladder, pancreas, oesophagus, stomach, colon, rectum, kidney, bladder, endometrium, cervix, adrenal gland, thyroid, brain, small intestine, large intestine, larynx, nasal cavity, throat cancer or neural tumours or testis and even more preferably an advanced malignant neoplasm.

The term “mammal” and “subject” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal Even more preferably, the mammal is a human.

In yet another further aspect, the present invention contemplates a pharmaceutical composition comprising the modulatory agent as hereinbefore defined and one or more pharmaceutically acceptable carriers and/or diluents. Said agents are referred to as the active ingredients

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding α-inhibin or a modulatory agent as hereinbefore defined. The vector may, for example, be a viral vector.

The present invention is further defined by the following non-limiting Examples.

Further features of the present invention are described in the following non-limiting Examples.

### EXAMPLE 1

### Evaluation of Prognostic Significance of Inhibin α Expression in Prostate Cancer

Study Subjects and Clinicopathological Data

A total of 174 subjects with peripheral-zone prostate cancer who had undergone radical prostatectomy at Stanford Medical Center between 1984 and 1992 was selected for the study. These were selected from an original group of 379 cases used for a previous study [Stamey et al. JAMA, 281: 1395-1400, 1999]. Of these 379, we excluded 72 cases whose cancers were 100% Gleason grade 3 as they had almost no incidence of recurrent disease in ≧5 years. 133 cases were excluded from further analysis of inhibin α expression because adequate tumor tissue was not available, blocks could not be located, or the quality of immunohistochemical staining was poor.

For all the subjects in the study, surgically removed prostates had been routinely subjected to a comprehensive histopathological review by a single pathologist as described previously [Stamey et al., J. Urol., 139: 1235-1241, 1988]. For each subject, an estimate was made of the percentage of the total cancer represented by grade 4 or 5 tumor [Stamey et al. 1999 (JAMA) supra] according to the Gleason system [Gleason et al., J. Urol., 111: 58-64, 1974]. Information on the subject's age at surgery and histopathological variables of prostate cancer was abstracted from an existing database. Information on race/ethnicity for individual patients had not been recorded in the database from which we abstracted patient information. In a previous study of 94 subjects from the same source population treated between 1991 and 1996, 95% of the patients were Caucasians.

PSA failure (or biochemical failure) was used as an outcome event for longitudinal analysis. Serum PSA levels following surgery were measured in the Stanford PSA Laboratory using the equimolar automated Tosoh A1A-600 assay (Foster City, Calif.), which was run in the ultrasensitive mode [Prestigiacomo et al., J. Urol., 152:1515-1519, 1994]. PSA tests performed in outside laboratories were obtained from patients by telephone interview. PSA failure was defined as having two consecutive PSA test results above a cutoff point (0.07 ng/ml for PSA levels measured by the sensitive Tosoh method and 0.2 ng/ml for measurements by less sensitive methods). When a patient had experienced PSA failure, time to failure was calculated as the number of months between the date of surgery and the date of the first of the two consecutive PSA tests that exceeded the cutoff point.

The study protocol was approved by the Panel on Medical Human Subjects at Stanford University.

Immunohistochemical Staining for Inhibin α

Sections (5 μm) were cut from formalin-fixed, paraffin-embedded tissues, deparaffinized in Hemo De (Fisher Scientific, Houston, Tex.) and dehydrated in Dehydrant 100 (VWR Scientific Products, Brisbane, Calif.). Endogenous peroxidase activity was blocked by incubation in methanol containing 3% hydrogen peroxide. Following three rinses in phosphate-buffered saline (PBS), tissue sections were treated with an antigen retrieval protocol. Sections were covered with 0.01 M citrate buffer, pH 6.0 (Biogenex, San Ramon, Calif.), and boiled in a microwave oven at high power for 4 minutes. After simmering for an additional 8 minutes at medium-low power, sections were removed from the microwave and cooled in the citrate buffer for 15 minutes at room temperature. The sections were rinsed in PBS, then incubated in CAS blocking reagent (Zymed, South San Francisco, Calif.) for 1 hour at room temp or overnight at 4° C. to block nonspecific labeling.

Sections were incubated with antibody PO #12 to detect the inhibin α subunit (90 μg/ml) as previously described and characterized [Robertson et al Mol Cell Endo 180: 79-86, 2001]. The Dako Envision System (Dako, Carpinteria, Calif.) was used to visualize labeling. After counterstaining with hematoxylin, sections were mounted and coverslipped.

Each immunostained tissue section was assessed by a single pathologist for the following: (i) overall staining quality (good, fair, or poor); (ii) staining intensity in tumor cells relative to nonmalignant cells in the same tissue section (+3, +2, +1, 0, −1, −2, or −3; positive scores indicate stronger staining in tumor cells compared to nonmalignant cells); (iii) overall variability of staining intensity within a tumor (minimal, mild, high, or equivocal); and (iv) variability of staining intensity between the Gleason grade 3 component and Gleason grade 4/5 component within a tumor (lower in grade 3, same, higher in grade 3, or equivocal). When the overall staining quality was judged to be poor, staining was repeated on another tissue section from the same or an alternative block from the same subject. If the repeat staining provided only poor-quality staining, the subject was excluded from further analysis.

Statistical Analysis

All statistical analyses were performed using the SAS statistical package, release 8.1 (SAS Institute, Cary, N.C.).

Differences in proportions between groups were tested by chi-square test or Fisher's exact test [Rosner, B. Fundamentals of biostatistics, Third ed. Boston: PWS-Kent Publishing Company, 1990]. Differences in means were tested by t-test for a comparison between two groups and by analysis of variance (ANOVA) for a comparison among three groups [Rosner, 1990 supra; SAS Institute. The GLM procedure. In SAS/STAT user's guide, release 6.03, pp. 549-640. Cary, N.C.: SAS Institute, 1988].

Relationship of inhibin α staining to the risk of PSA failure was examined by Cox proportional hazards analysis with the use of the PHREG procedure with the “exact” option for handling ties [SAS Institute. The PHREG procedure. In SAS/STAT software: changes and enhancements through release 6.12, pp. 873-948. Cary, N.C.: SAS Institute, 1997]. The Cox model included the maximum tumor intensity (MT]) score of inhibin a staining as a main covariate of interest and hazard ratios (HRs) and their 95% confidence intervals (CIs) were estimated. For inhibin α staining, MTI scores of +2 and +3, respectively, were compared with an MTI score of −1 to 1 as a reference category. All HRs were adjusted for age at surgery, and some HRs were further adjusted for tumor grade (% Gleason grade 4/5) and cancer volume.

Those men who had elevated serum PSA levels immediately after their surgery were considered failed at the time of surgery (i.e., never cured). Four subjects had a serum PSA level above the cutoff point only in their last follow-up PSA test available, and thus we could not confirm their failure status definitively in the absence of a subsequent PSA test also exceeding the cutoff point. To test how the failure status of these four subjects affected the results of statistical analysis, longitudinal analysis for PSA failure were performed in two ways, regarding the four subjects with indeterminate failure status as: (a) experiencing PSA failure or (b) not experiencing PSA failure.

Results

Patient and Tumor Characteristics

Table 2 summarizes characteristics of the 174 originally selected study subjects and their prostate cancers by the availability of α-inhibin subunit immunohistochemical data. No statistically significant difference was observed for age, tumor grade, cancer volume, preoperative serum PSA level, or proportion of PSA failures between the subjects whose tumors yielded acceptable immunohistochemical data for α-inhibin subunit and those who were excluded due to lack of adequate tissue or suboptimal staining.

For the 107 subjects with inhibin α subunit data, the median follow-up after radical prostatectomy was 41.2 months (range 0-144.2 months). Twenty-one men (19.6%) had elevated serum PSA levels immediately after their surgery and were considered failed at the time of surgery (i.e., never cured) in the longitudinal analysis. An additional 35 men (32.7%) experienced PSA failure between 2.9 and 94.1 months (median 30.4 months) after surgery. Four other men (3.7%) presented an elevated serum PSA level in their latest PSA test available; these subjects were regarded as either failure or non-failure in alternative statistical analyses.

Inhibin α Expression and Prognostic Significance

Of the 107 tumors that yielded an acceptable quality of inhibin α subunit staining, only 2 (1.9%) showed less intense staining than nonmalignant prostate glands from the same subject (i.e., MTI score=−1). In 10 subjects (9.4%), the staining intensity for α-inhibin subunit was about the same between cancer and non-cancer tissues (MTI=0). In the remaining 95 subjects (88.8%), tumors showed stronger inhibin α subunit staining than non-tumor tissues: MTI scores of +1, +2, and +3 were observed in 20 (18.7%), 48 (44.9%), and 27 (25.2%) of subjects, respectively.

Table 3 presents the results of Cox proportional hazards analysis for inhibin α subunit staining and PSA failure. Alternative models were run with or without adjustment for tumor grade (% Gleason grade 4/5) and cancer volume while the failure status of the four subjects whose serum PSA levels were elevated only in their latest PSA test available was considered as either non-failure or failure. Compared with subjects whose prostate tissue presented little difference in inhibin α subunit staining intensity between tumor and non-tumor cells (i.e., MTI score=−1 to 1), those whose cancer showed the most elevated expression of inhibin α subunit (i.e., MTI score=3) had higher risk of PSA failure (with HR estimates ranging from 1.43 to 1.94 depending on models used) although the risk elevation was not statistically significant.

### EXAMPLE 2

### Summary of Inhibin α Detection with the PO #12 Antibody on Human Prostate Cancer Biopsy Samples

32 biopsy samples from 28 prostate cancer patients were stained with the PO #12 antibody directed to amino acids 73-96 of the αC region using a DAKO autostainer. In these 32 samples both benign glands and malignant epithelial cells were present in the same biopsy sample or in a different biopsy from the same patient. The score for staining intensity are as follows: − negative stain; + normal intensity staining; ++ strong staining; +++ very strong staining. The results shown in table 3 compare the intensity of α-inhibin −12 staining between benign gland and malignant epithelial cells in the same biopsy or an area from the same patient. As shown in table 4, 18 patients had increased inhibin-α intensity in prostate cancer compared to BPH (56.25%), while 13 patients had similar inhibin-α intensity in both prostate cancer and BPH (40.62%).

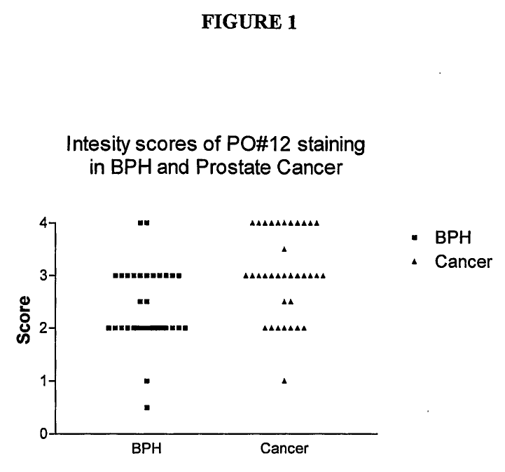

FIG. 1 was plotted using the data as score (−=1, +=2, ++=3, +++=4) and demonstrates a greater proportion of inhibin-α in prostate cancer compared to BPH glands.

### EXAMPLE 3

### Evaluation of Functional Significance of Inhibin α Expression in Non-Aggressive vs Highly Aggressive Prostate Cancer Cell Lines

Cell Lines

Human prostate tumor epithelial cell lines LNCaP and PC3, were obtained from American Type Culture Collection (Rockville, Md., USA). Cell lines were routinely cultured in Dulbecco's Modified Eagle's Medium (DMEM; Gibco. NY, USA) with 10% (v/v) heat-inactivated foetal calf serum (FCS) (PA Biologicals Co. Pty Ltd, NSW, Australia) and antibiotics (100 UI/ml penicillin and 10 μg/ml streptomycin; CSL Ltd, Parkville, Vic, Australia) in 75 cm² culture flasks (Costar; Corning Costar Corp., Cambridge, Mass., USA) at 37° C. in a humidified atmosphere of 5% CO₂ in air. Cell lines were passaged every four days by trypsinisation.

Preparation of INHA Expression Vector for Transfection

Human INHA cDNA subcloned into pcDNA3.1 and pcDNA3.1 vector were purchased from Invitrogen, (Carisbad, Calif., USA). Both plasmids were introduced into competent cells by heat stock and zeocin resistant colonies were selected and propagated. The plasmids were purified using HiSpeed™ Plasmid Maxi Kit, (Qiagen, Clifton Hill, Victoria, Australia) and linearised by Pme 1 restriction enzyme. Upon complete digestion, the DNA was extracted using ethidium bromide/High Salt extraction method. Briefly, 15 μl of 10 mg/ml ethidium bromide (EtBr) was added to the digestion mix followed by addition of 140 μl of 3M sodium acetate pH 5.2. After mixing, equal volume of Tris-saturated phenol/chloroform was added, mixed well and centrifuged for 2 minutes at maximum speed. The upper phase was removed into a new tube and extracted twice with chloroform. Finally, the upper phase was extracted twice with 1 ml of butanol. The lower phase was then incubated in 100% ethanol for 2 minutes at room temperature, centrifuged for 20 mins, washed in 70% ethanol and resuspended in 50 μl of 1× TE buffer (Qiagen, Clifton Hill, Victoria, Australia). The resuspended DNA was then microdialyzed on a microdialysis filter paper (Millipore, Bedford, Mass.) for 2 hrs on 1× TE pH7.6 (10 mM Tris-Cl, 1 mM EDTA).

Stable Transfection of LNCaP and PC3 Cell Lines

LNCaP cells seeded at 6.24×10⁵ cells/well in a 6-well plate were cultured (50-80% confluence) for 24 hours. Lipofectamine plus (Invitrogen, Carisbad, Calif., USA) was then used for transfections according to the manufacturer's instructions. Briefly, the cells were transfected with 3.84 μg plasmid DNA using 4 μl Lipofectamine and 19.2 μl Lipofectamine plus. After 3 hrs, the transfection media was replaced with DMEM+10% FCS, and after 48 hrs, media was replaced with selection media (DMEM supplemented with 10% FCS and 360 μg/ml zeocin).

PC3 cells seeded at 4.8×10⁵ cells/well in a 6-well plate were cultured (50-80% confluence) for 24 hours. Superfect (Qiagen Pty Ltd, Clifton Hill, Victoria, Australia) was then used for transfections according to the manufacturer's instructions. Briefly, the cells were transfected with 8.64 μg plasmid DNA using 14.4 μl of Superfect. After 3 hrs, the transfection media was replaced with DMEM+10% FCS, and after 48 hrs, media was replaced with selection media (DMEM supplemented with 10% FCS and 360 μg/ml zeocin).

Individual colonies surviving after 2-3 weeks selection were picked and propagated in DMEM supplemented with 10% FCS and 360 μg/ml zeocin. Integration of the plasmids was confirmed by genomic PCR. Expression of inhibin α from the plasmids was confirmed by RT-PCR and Western blot with RI monoclonal antibody raised to the mature αC region of the inhibin α subunit.

Growth Rate

Cells were seeded at a density of 5×10⁴ cells/well in a 24-well plate in DMEM medium supplement with 100 μg/ml zeocin and 10% FCS and incubated at 37° C. in 5% CO₂. Triplicate wells were harvested by trypsinisation on day 1, 2, 3,4, and 5, and numbers of cells/well were counted by haemocytometer. Each experiment was repeated twice.

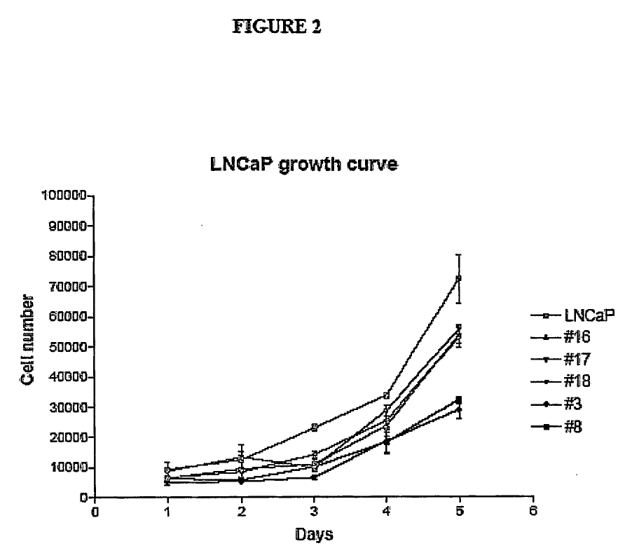

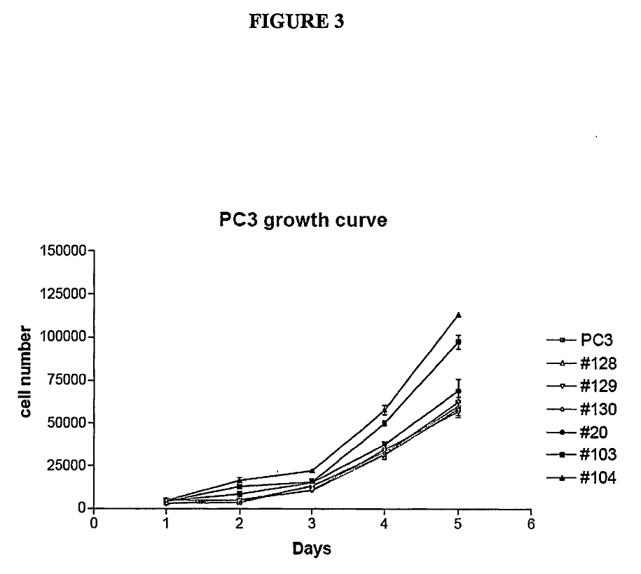

Inhibin α over-expressing LNCaP cells grew slower than negative control LNCaP cells that did not express inhibin α (FIG. 2). In contrast, inhibin α over-expressing PC3 cells grew faster than the negative control PC3 cells that did not express inhibin α (FIG. 3).

[³H] Thymidine Incorporation Assay for DNA Synthesis

LNCaP and PC3 cells were plated at a density of 5000 cells/well and 2500 cells/well, respectively, in DMEM with 10% FCS and 100 μg/ml into 96-well plates for 48 hrs. [³H] thymidine (0.5 uCi/ml) (Amersham Pharmacia Biotech, UK Ltd, Little Chalfont, UK) prepared in DMEM only was added to the cells for 20 hrs, after which the cells were harvested using a micromate 196 Cell Harvester (Packard Instrument Co. Merdien, Conn.) and levels of [³H] thymidine incorporation were determined. Each experiment was repeated twice.

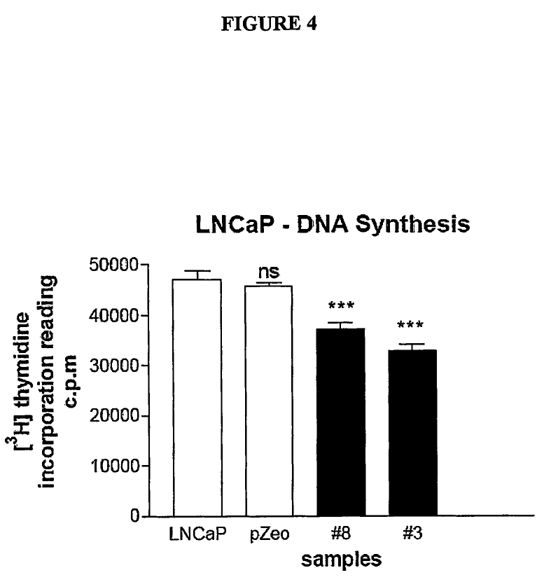

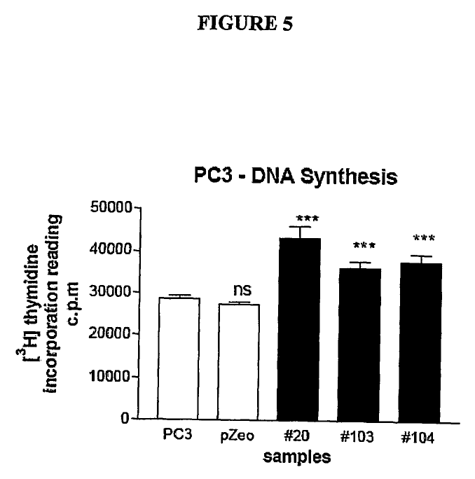

Inhibin α over-expressing LNCaP cells showed significantly reduced DNA synthesis compared to negative control LNCaP cells that did not express inhibin α (p<0.05; FIG. 4). In contrast, inhibin α over-expressing PC3 cells showed significantly increased DNA synthesis compared to the negative control PC3 cells that did not express inhibin α (p<0.05; FIG. 5).

### EXAMPLE 4

### Inhibin α Expression in Gleason Grade 3 and 4 Prostate Cancer

Radical prostatectomy tissues from 20 patients with localised prostate cancer were selected. The patients had undergone surgical removal of the prostate and the cancer was confirmed histopathologically to be confined within the prostatic capsule.

Immunostaining for inhibin α PO#12 monoclonal antibody directed to the mature αC region of the inhibin α subunit [Robertson et al., 2001 supra] was performed by DAKO Autostainer (DAKO, Carpinteria, USA). The sections were dewaxed and rehydrated and antigen retrieval was performed in 0.01 M citrate buffer pH 6.0 by microwave for 3 minutes at high power, 5 minutes for low power and then cooled down for 20 minutes. On the autostainer, sections were incubated with 0.03% H₂O₂ (DAKO, Carpinteria, USA) for 15 minutes and then with CAS blocking solution (Zymed, San Francisco, Calif., USA) for 10 minutes. The sections were incubated with 5 μg/ml of the inhibin PO#12 antibody for 2 hours. The antibody was detected by incubation with Envision polymer-anti-mouse-HRP (DAKO, Carpinteria, USA) for 15 minutes and visualised by reaction with diaminobenzidine (DAB; DAKO, Carpinteria, USA) for 5 minutes. Sections were counterstained by Hematoxylin (DAKO, Carpinteria, USA ). Inhibin a staining intensity was recorded by histopathologists in benign secretory and basal epithelia, benign stroma and tumour cells. Intensity was scored as no staining=1, variable±staining=2, 1+staining=3, 2+staining=4, 3+staining=5.

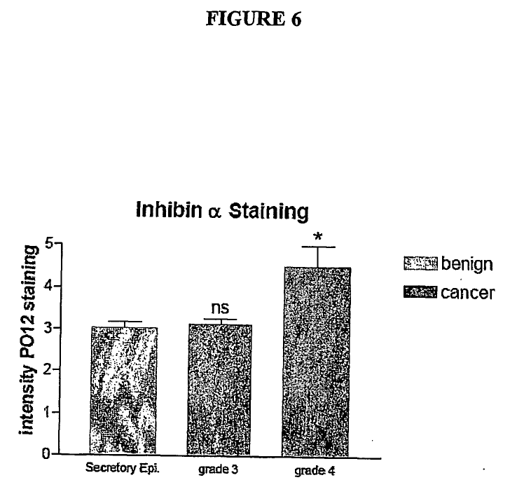

Inhibin α immunostaining was significantly increased in Gleason grade 4 cancers compared to Gleason grade 3 cancers. There was no significant difference in inhibin a immunostaining between benign secretory epithelia and Gleason grade 3 staining within the same sections (FIG. 6).

### EXAMPLE 5

### Inhibin α Expression in Cribriform Prostate Cancers

Immunohistochemistry was performed on radical prostatectomy tissue from 28 prostate cancer patients with Gleason Grade sum of greater than or equal to 7. Sections were immunostained as described above. Inhibin a staining intensity was recorded by histopathologists as no staining=1, variable±staining=2, 1+staining=3, 2+staining 4, 3+staining=5.

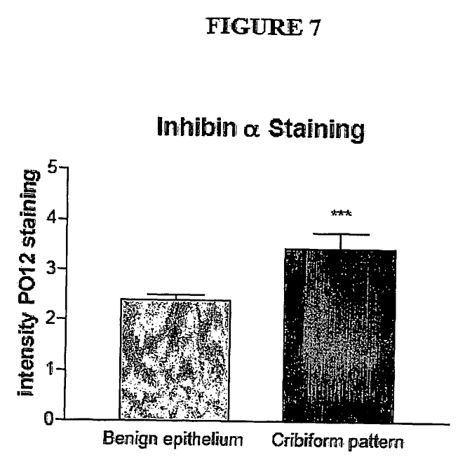

Inhibin α immunostaining was significantly increased in cribriform cancers compared to benign secretory epithelium (p<0.0005; FIG. 7).

### EXAMPLE 6

### Inhibin α Expression in Cancers Other than Prostate Cancer

Immunohistochemistry was performed on human normal tissue array (AA8) and a various human cancers tissue array (BB4), each containing 60 tissues per slide (SuperBioChips Laboratories, Seoul, Korea). Sections were immunostained with R1 and PO12 antibodies as described above.

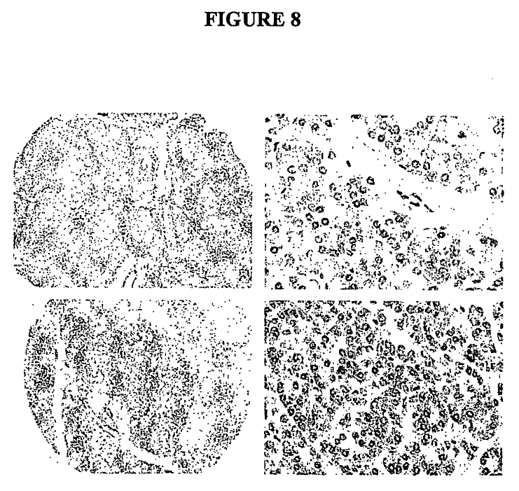

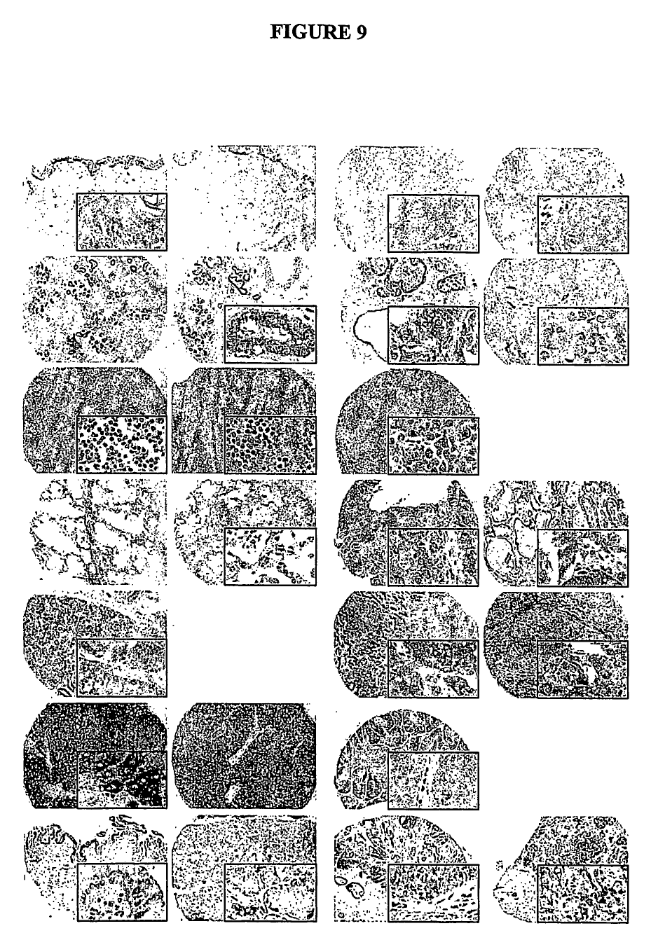

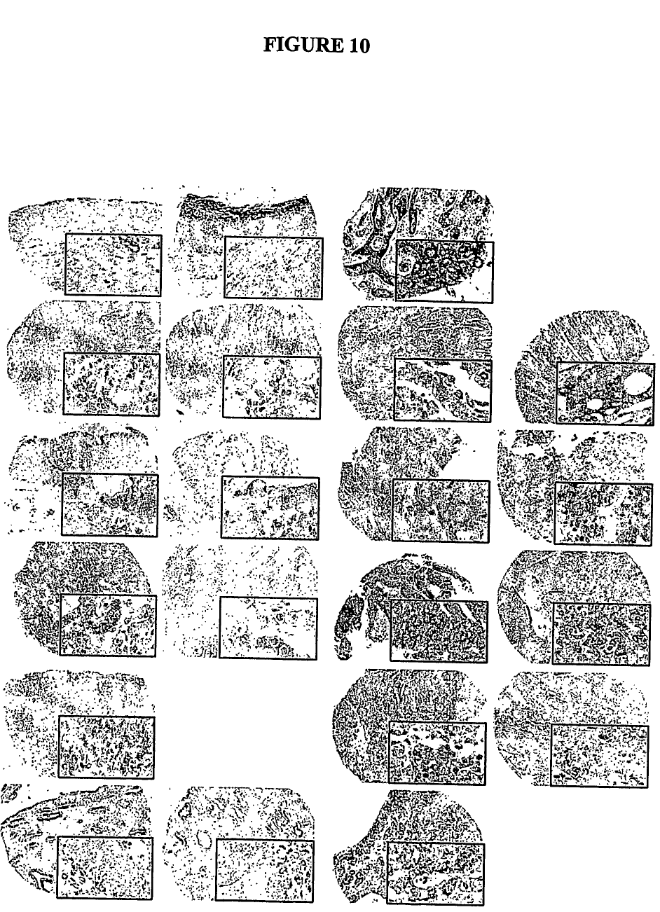

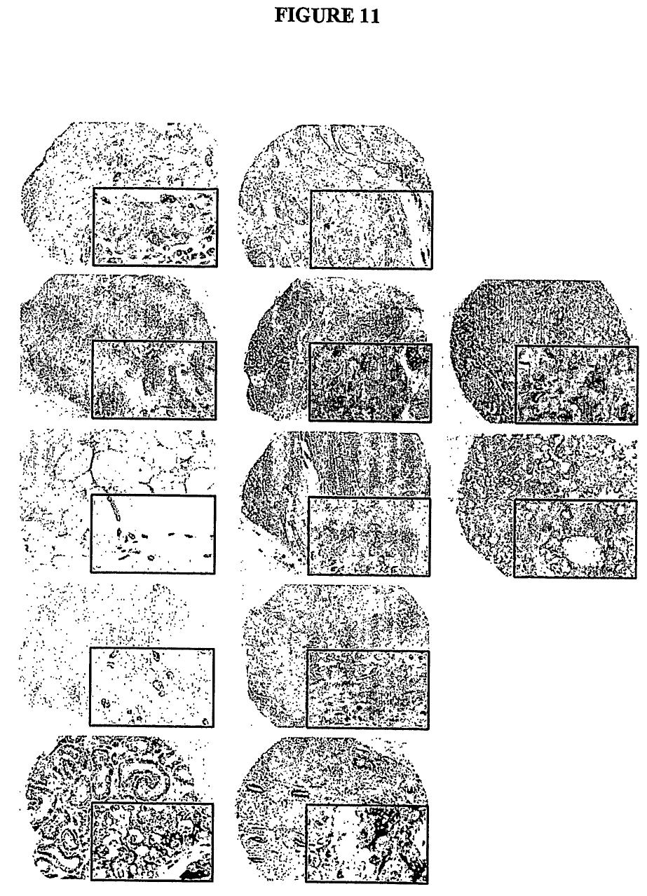

Inhibin α staining with the R1 antibody was rarely observed in either normal tissues or cancers, with the notable exception of pancreatic adenocarcinoma (FIG. 8d). In contrast, inhibin α staining with the PO12 antibody was observed in a wide range of normal and malignant human tissues. Generally, cytoplasmic staining was observed and occasionally nuclear localisation was also observed. Differences in inhibin α immunostaining between normal and equivalent malignant tissues was regularly observed. FIGS. 9 to 11 fully describe the staining pattern and the descriptions below indicate some of the significant findings.

The pancreas showed an increase in inhibin α immunostaining in malignancy compared to normal tissue using both R1 and PO12 antibodies (FIG. 8a-d and FIG. 9y and z2).

Other tissues that demonstrated an increase in inhibin α immunostaining in malignant cells compared to normal or benign cells using the PO12 antibody included skin (FIG. 9a-d), lymph node (FIG. 9i-k), lung (FIG. 9l-p), salivary glands (FIG. 9q-s), gallbladder (FIG. 9x,z), pancreas (FIG. 9y,z2), esophagus (FIG. 10a-c), stomach (FIG. 9d-g), colon (FIG. 10h,j), rectum (FIG. 10i,k), kidney (FIG. 10l-o), uterine cervix (FIG. 11a,b), thyroid (FIG. 11f-h) and brain (FIG. 11i,j). These results are consistent with the hypothesis that inhibin α is upregulated in advanced cancers that are likely to become, or have become, metastatic.

Tissues that demonstrated a decrease in inhibinα immunostaining or variable immunostaining for inhibin-α in malignant cells compared to normal or benign cells using the PO12 antibody included breast (FIG. 9e-h) and liver (FIG. 9t-v), urinary bladder (FIG. 10p-r), endometrium (FIG. 10s-u), adrenal gland (FIG. 11c-c) and testis (FIG. 11k,l). These results are consistent with the hypothesis that inhibin-α is downregulated in early stage cancers that have not yet acquired aggressive or metastatic properties, prior to an upregulation of inhibin-α in advanced cancers that are likely to become, or have become, metastatic.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 2
Characteristics of originally selected study subjects with prostate
cancer by availability of inhibin α immunohistochemistry data

| | Inhibin α data | | |
|---|---|---|---|
| | Available | Not available | |
| Variable | (N = 107) | (N = 67) | P-value^{a} |
| Age at surgery (years)^{b} | 65.4 ± 5.9 | 63.9 ± 7.2 | 0.14 |
| % Gleason grade 4/5^{b} | 44.1 ± 26.3 | 42.1 ± 26.4 | 0.64 |
| Cancer volume (cm³)^{b} | 6.46 ± 7.06 | 5.50 ± 5.76 | 0.35 |
| Preoperative serum PSA | 23.6 ± 28.6 | 20.4 ± 20.5 | 0.40 |
| level (ng/ml)^{b} | | | |
| Number of Subjects with | | | |
| PSA failure | | | |
| Failure at time of surgery | 21 (19.6%) | 10 (14.9%) | 0.43 |
| Definitive failure during | 35 (32.7%) | 26 (38.8%) | 0.41 |
| follow-up | | | |
| Possible failure | 4 (3.7%) | 0 (0%) | 0.30 |

^{a}For the difference between the two groups by t-test for continuous variables and by chi-square test for the number of subjects with PSA failure, except that Fisher's exact test was used for “possible failure”.
^{b}Mean ± standard deviation.

TABLE 3
Results of the Cox proportional hazards model analysis for inhibin α^{a}

| | “Possible” PSA | Hazard ratio | MTI^{c} = −1 to 1 | MTI = 2 | | MTI = 3 | |
|---|---|---|---|---|---|---|---|
| Model | failure considered as | adjusted for^{b} | HR^{c} | HR | 95% CI^{c} | HR | 95% CI |
| 1 | Failure | Age | 1.00 (Reference) | 1.28 | 0.68-2.40 | 1.61 | 0.77-3.37 |
| 2 | Failure | Age, tumor grade, | 1.00 (Reference) | 0.94 | 0.48-1.85 | 1.43 | 0.66-3.13 |
| | | and cancer volume | | | | | |
| 3 | Non-failure | Age | 1.00 (Reference) | 1.39 | 0.71-2.72 | 1.94 | 0.90-4.19 |
| 4 | Non-failure | Age, tumor grade, | 1.00 (Reference) | 1.04 | 0.51-2.11 | 1.71 | 0.76-3.84 |
| | | and cancer volume | | | | | |

^{a}Based on 107 subjects for whom acceptable inhibin α staining was available. Failure at time 0 included.
^{b}The following categorical variables were used for adjustment: age at surgery (in years) <60, 60-64, 65-69, 70+; tumor grade (% grade 4/5) <20%, 20-39%, 40-69%, ≧70%; and cancer volume (in cc) <2.57, 2.57-4.53, 4.54-7.76, 7.77+ (quartiles).
^{c}MTI, maximum tumor intensity of inhibin α staining (positive scores indicate stronger staining in tumor cells compared with nonmalignant cells); HR, hazard ratio; CI, confidence interval.

TABLE 4
The change of the intensity of PO #12 antibody staining
in the prostate cancer epithelium and BPH gland

| | Intensity | Intensity | Intensity | |
|---|---|---|---|---|
| | Pca > BPH | Pca = BPH | Pca < BPH | Total |
| Number | 18 | 13 | 1 | 32 |
| Percentage % | 56.52 | 40.62 | 3.13 | 100 |

### BIBLIOGRAPHY

Balanathan B., Ball E. M. A., Wang H., Harris S. E., Shelling A. N. and Risbridger, G. L., Epigenetic regulation of inhibin α-subunit gene in prostate cancer cell lines, Journal of Molecular Endocrinology (2004) 32, 55-67
Cipriano S. C., Chen, L., Kumar, T. R. and Matzuk, M. M. (2000) ‘Follistatin is a modulator of gonadal tumor progression and the activin-induced wasting syndrome in inhibin-deficient mice’, Endocrinology, 141, 2319-27
Gleason, D. F. and Mellinger, G. T. J. Urol., 111: 58-64, 1974.
Cohen, R. J., McNeal, J. E. and Baillie, T. (2000) Prostate, 43: 11-9
Dalkin, A. C., Gilrain, J. T., Bradshaw, D. and Myers, C. E. (1996) Endocrinology, 137: 5230-5
Fisher, J. L., Schmitt, J. F., Howard, M. L., Mackie, P. S., Choong, P. F. and Risbridger, G. P. (2002) Cell Tissue Res, 307: 337-45
Lewis, K. A., Gray, P. C., Blount, A. L., MacConell, L. A., Wiater, E., Bilezikjian, L. M. and Vale, W. (2000) Nature, 404: 411-4
Lopez, P., Vidal, F., Rassoulzadegan, M. and Cuzin, F. (1999) ‘A role of inhibin as a tumor suppressor in Sertoli cells: down- regulation upon aging and repression by a viral oncogene’, Oncogene, 18, 7303-9
Matzuk, M. M., Finegold, M. J., Su, J. G., Hsueh, A. J. and Bradley, A. (1992) ‘Alpha-inhibin is a tumour-suppressor gene with gonadal specificity in mice’, Nature, 360, 313-9
Matzuk, M. M., Finegold, M. J., Mather, J. P., Krummen, L., Lu, H. and Bradley, A. (1994) ‘Development of cancer cachexia-like syndrome and adrenal tumors in inhibin-deficient mice’, Proc Natl Acad Sci USA, 91, 8817-21
Matzuk, M. M., Kumar, T. R., Shou, W., Coerver, K. A., Lau, A. L., Behringer, R. R. and Finegold, M. J. (1996) ‘Transgenic models to study the roles of inhibins and activins in reproduction, oncogenesis, and development’, Recent Prog Horm Res, 51, 123-54
Matzuk, M. M. and Bradley, A. (1994) ‘Identification and analysis of tumor suppressor genes using transgenic mouse models’, Semin Cancer Biol, 5, 37-45
McNeal, J. E. and Yemoto, C. E. (1996) Am J Surg Pathol, 20: 802-14
McPherson, S. J., Mellor, S. L., Wang, H., Evans, L. W., Groome, N. P. and Risbridger, G. P. (1999) Endocrinology, 140: 5303-5309
McPherson, S. J., Thomas, T. Z., Wang, H., Gurusinghe, C. J. and Risbridger, G. P. (1997) J Endocrinol, 154: 535-545
Mellor, S. L., Richards, M. G., Pedersen, J. S., Robertson, D. M. and Risbridger, G. P. (1998) ‘Loss of the expression and localization of inhibin alpha-subunit in high grade prostate cancer’, J Clin Endocrinol Metab, 83, 969-975
Moore, A., Basilion, J., Chiocca, E., and Weissleder, R., BBA, 1402:239-249, 1988.
Pangas, S. A. and Woodruff, T. K. (2000) ‘Activin Signal Transduction Pathways’, Trends Endocrinol Metab, 11, 309-314
Prestigiacomo, A. F. and Stamey, T. A. J. Urol., 152: 1515-1519, 1994.
Robertson et al Mol Cell Endo 180: 79-86, 2001.
Robertson, D. M., Stephenson, T., Cahir, N., Tsigos, A., Pruysers, E., Stanton, P. G., Groome, N. and Thirunavukarasu, P. (2001) Mol Cell Endocrinol, 180: 79-86
Rosner, B. Fundamentals of biostatistics, Third ed. Boston: PWS-Kent Publishing Company, 1990.
Rubin, M. A., de La Taille, A., Bagiella, E., Olsson, C. A. and O'Toole, K. M. (1998) Am J Surg Pathol, 22: 840-8
SAS Institute. The GLM procedure. In SAS/STAT user's guide, release 6.03, pp. 549-640. Cary, N.C.: SAS Institute, 1988.
SAS Institute. The PHREG procedure. In SAS/STAT software: changes and enhancements through release 6.12, pp. 873-948. Cary, N.C.: SAS Institute, 1997.
Schmitt, J. F., Millar, D. S., Pedersen, J. S., Clark, S. L., Venter, D. J., Frydenberg, M., Molloy, P. L. and Risbridger, G. P. (2002) ‘Hypermethylation of the inhibin alpha-subunit gene in prostate carcinoma’, Mol Endocrinol, 16, 213-20
Stamey, T. A., McNeal, J. E., Freiha, F. S., and Redwine, E. J. Urol., 139: 1235-1241, 1988.
Stamey, T. A., McNeal, J. E., Yemoto, C. M., Sigal, B. M., and Johnstone, I. M. JAMA, 281: 1395-1400, 1999.
Thomas, T. Z., Wang, H., Niclasen, P., O'Bryan, M. K., Evans, L. W., Groome, N. P., Pedersen, J. and Risbridger, G. P. (1997) ‘Expression and localization of activin subunits and follistatins in tissues from men with high grade prostate cancer’, J Clin Endocrinol Metab, 82, 3851-9
Thomas, T. Z., Chapman, S. M., Hong, W., Gurusingfhe, C., Mellor, S. L., Fletcher, R., Pedersen, J. and Risbridger, G. P. (1998) ‘Inhibins, activins, and follistatins: expression of mRNAs and cellular localization in tissues from men with benign prostatic hyperplasia’, Prostate, 34, 34-43
Vale, W., Hseuh, A., Rivier, C. and Yu, J. (1990) In Peptide growth factors and their receptors: Handbook of Experimental Physiology, Vol. 95 (Fds, Sporn, M. and Roberts, A.) Springer-Verlag, Berlin, pp. 211-248
Wang, Q. F., Tilly, K. I., Tilly, J. L., Preffer, F., Schneyer, A. L., Crowley, W. F., Jr. and Sluss, P. M. (1996) Endocrinology, 137: 5476-83
Wedemeyer, N., Potter, T., Wetzlich, S. and Gohde, W. Clinical Chemistry 48:9 1398-1405, 2002
Weissleder, R., Moore, A., Ph.D., Mahmood-Bhorade, U., Benveniste, H., Chiocca, E. A., Basilion, J. P. Nature Medicine, 6:351-355, 2000
Wilcox, G., Soh, S., Chakraborty, S., Scardino, P. T. and Wheeler, T. M. (1998) Hum Pathol, 29: 1119-23

1. A method of detecting the onset of an advanced neoplasm or a predisposition to developing an advanced neoplasm in a mammal said method comprising screening for the level of inhibin protein and/or gene expression in a biological sample derived from said mammal wherein an increase in the level of inhibin and/or gene expression is indicative of the onset of an advanced neoplasm or a predisposition to developing an advanced neoplasm.

2. A method of monitoring for the onset or progression of an advanced neoplasm in a mammal said method comprising screening for the modulation in the level of inhibin in a biological sample derived from said mammal wherein the level of said inhibin relative to the normal level of inhibin is indicative of the onset or progression of an advanced neoplasm.

3. The method according to claim 1 or 2 wherein the biological sample is selected from the group including serum, tissue extract, body fluids, cell culture medium, extracellular medium, supernatants, biopsy specimens or resected tissue.

4. The method according to claim 3 wherein said advanced neoplasm is an advanced malignant neoplasm.

5. The method according to claim 4 wherein said advanced malignant neoplasm is a metastatic neoplasm.

6. The method according to claim 1 or 2 wherein said inhibin is α-inhibin.

7. The method according to claim 6 wherein said α-inhibin is the αC region of the α-inhibin protein.

8. The method according to claim 7 wherein said αC region comprises amino acids 73-96 of the αC region.

9. The method according to claim 6 wherein said α-inhibin is the α-inhibin protein and said protein is detected utilising the monoclonal antibody PO#12.

10. The method according to claim 1 or 2 wherein said neoplasm is a neoplasm of the prostate, skin, breast, lymiph node, lung, salivary gland, liver, gall bladder, pancreas, oesophagus, stomach, colon, rectum, kidney bladder, cervix, adrenal gland, thyroid, brain, testis or with the endometrium.

11. (canceled)

12. The method according to claim 10 wherein said skin neoplasm is a melanoma or a squamous cell carcinoma, said breast neoplasm is an invasive papillary carcinoma or an infiltrating breast carcinoma, said lymph node neoplasm is a lymphoma, said lung neoplasm is a squamous cell carcinoma or lung adenocarcinoma, said salivary gland neoplasm is a pleomorphic adenoma of the parotid gland or a salivary duct carcinoma, said liver neoplasm is a hepatocellular carcinoma, said gall bladder neoplasm, pancreatic neoplasm, stomach neoplasm, colon neoplasm or rectal neoplasm is an adenocarcinoma, said oesophazial neoplasm is a squamous cell carcinoma, said kidney neoplasm is a transitional cell carcinoma or a renal cell carcinoma, said bladder neoplasm is a carcinoma of the bladder or a transitional cell carcinoma of the bladder, said endometrial neoplasm is an endometrial carcinoma, said cervical neoplasm is a squamous cell carcinoma, said adrenal gland neoplasm is an adrenal cortical carcinoma or an adrenal pheochromocztoma, said thyroid neoplasm is a thyroid papillary carcinoma or an invasive follicular carcinoma of the thyroid, said brain neoplasm is a meningioma, or said testis neoplasm is a testis seminoma.

13.-50. (canceled)

51. A diagnostic kit for assaying biological samples comprising an agent for detecting α-inhibin protein or encoding nucleic acid molecule and reagents useful for facilitating the detection by the agent in the first compartment.

52. The kit according to claim 51 wherein said agent is an antibody directed to α-inhibin protein.

53. The kit according to claim 52 wherein said antibody is a monoclonal antibody.

54. The kit according to claim 53 wherein said monoclonal antibody is PO#12.

55. A method of modulating the invasiveness of a neoplastic cell, said method comprising modulating the level of intracellular inhibin protein wherein up-regulating inhibin levels to a functionally effective level induces said invasiveness and down-regulating inhibin levels to a functionally ineffective level inhibits said invasiveness.

56. The method according to claim 55 wherein said neoplastic cell is a cell of the skin, lymph node, lung, salivary gland, liver, gallbladder, pancreas, cervix, brain, oesophagus, stomach, colon, rectum, kidney, bladder, small intestine, large intestine, larnx, nasal cavity, throat, neural tissue, endometrium, adrenal gland, thyroid, brain, testis, breast or prostate.

57.-60. (canceled)

61. A method for the treatment and/or prophylaxis of a condition characterized by an advanced neoplasm or a predisposition to the development of a condition characterized by an advanced neoplasm in a mammal, said method comprising modulating the level of intracellular inhibin wherein down-regulating said inhibin levels to a functionally ineffective level inhibits invasiveness.

62. The method according to claim 61 wherein said neoplasm is a neoplasm of the skin, lymph node, lung, salivary gland, liver, gallbladder, pancreas, cervix, brain, oesophagus, stomach, colon, rectum, kidney, bladder, small intestine, large intestine, larynx, nasal cavity, throat, neural tissue, endometrium, adrenal gland, thyroid, testis, breast or prostate.

63.-66. (canceled)

67. The method according to claim 55 wherein said neoplastic cell is a malignant neoplastic cell.

68. The method according to claim 67 wherein said malignant neoplastic cell is a metastatic neoplasm.

69. The method according to claim 55 or 61 wherein said inhibin is α-inhibin.

70. The method according to claim 55 wherein said modulation is downregulation of inhibin and said downregulation is achieved by contacting said neoplastic cell with a proteinaceous or non-proteinaceous molecule with functions as an antagonist to the inhibin expression product.

71. The method according to claim 70 wherein said molecule is an antibody.

72. The method according to claim 71 wherein said antibody is PO#12.

73. The method according to claim 55 wherein said modulation is achieved by contacting said neoplastic cell with a proteinaceous or non-proteinaceous molecule which modulates transcriptional and/or translational regulation of the inhibin-α gene.

74. The method according to claim 55 wherein said modulation is upregulation of inhibin levels and said upregulation is achieved by contacting said cell with a proteinaceous or non-proteinaceous molecule which finctions as an agonist of the inhibin expression product.

75. The method according to claim 55 wherein said modulation is upregulation of inhibin and said upregulation is achieved by introducing into said cell a nucleic acid molecule encoding inhibin or functional equivalent, derivative or homologue thereof or the inhibin expression product or functional derivative, homologue, analogue, equivalent or mimetic thereof.

76.-77. (canceled)

78. A pharmaceutical composition comprising an agent capable of modulating the functionally effective level of inhibin together with one or more pharmaceutically acceptable carriers and/or diluents when used in the method of claim 55.
